(19) United States
(12) Patent Application Publication
(10) Pub. No.: US 2021/0000806 A1
(43) Pub. Date: Jan. 7, 2021

(54) COMPOSITIONS AND METHODS FOR TREATING INFLAMMATORY BOWEL DISEASE AND FUSOBACTERIA-CAUSED OR RELATED DISEASES AND CONDITIONS

(71) Applicant: Thomas Julius BORODY, Five Dock (AU)

(72) Inventor: Thomas Julius BORODY, Five Dock (AU)

(21) Appl. No.: 17/040,881

(22) PCT Filed: Mar. 25, 2019
(86) PCT No.: PCT/AU2019/050263

§ 371 Date: Sep. 23, 2020

Related U.S. Application Data
(60) Provisional application No. 62/647,384, filed on Mar. 23, 2018.
(60) Provisional application No. 62/688,653, filed on Jun. 22, 2018.

(51) Int. Cl. A61K 31/437; A61K 31/4164; A61P 1/04; A61K 31/65; A61K 31/496; A61K 31/426; A61K 9/00; A61K 31/665; A61K 45/06; A61K 47/36; A61K 47/46; A61K 47/32; A23L 33/10

(52) U.S. Cl. CPC A61K 31/437; A61K 31/4164; A61P 1/04; A61K 31/65; A61K 31/496; A61K 31/426; A23V 2002/00; A61K 31/665; A61K 45/06; A61K 47/36; A61K 47/46; A61K 47/32; A23L 33/10; A61K 9/0056

(57) ABSTRACT

Provided herein are pharmaceutical compositions, therapeutic combinations, devices and methods for treating, ameliorating, reversing, causing the remission of, and/or preventing (acting as a prophylaxis, or preventing the initiation of) an inflammatory bowel disorder (IBD) or inflammatory bowel disease (IBD), Ulcerative Colitis; Crohn's disease; J-pouch; fistulising Crohn's disease; a Colitis which can be microscopic, lymphocytic or collagenous; an eosinophilic colitis; indeterminate colitis; idiopathic colitis; diverticulosis and diverticulitis; relapsing diverticulitis; constipation associated inflammatory bowel disease and/or small intestinal bacterial overgrowth; Irritable Bowel Syndrome (IBS) with or without diarrhoea, constipation or pain predominant IBS; periodontitis; rheumatoid arthritis; respiratory infections, appendicitis, vascular disorders such as thrombophlebitis; bacteremia; osteomyelitis; septic shock; Alzheimer's disease; Lemierre syndrome (postanginal sepsis); colonic polyps or adenomas (optionally hyperplastic, adenomatous or serrated adenomas) or preventing the growth of colonic polyps or adenomas, bowl cancer, or metastases (optionally preventing the initiation or promotion of bowl cancer or metastasis); pharyngitis; otitis; sinusitis; and any disease, symptom or condition caused or exacerbated by a Fusobacteria (optionally, a F. nucleatum or F. varium) infection. In alternative embodiments, pharmaceutical compositions comprise rifaximin alone or in combination with other antibiotics or drugs.

### FIELD

This invention generally relates to medicine and gastroenterology, pharmacology and microbiology. In alternative embodiments, provided are pharmaceutical compositions, therapeutic combinations, devices and methods for treating, ameliorating, reversing (e.g., causing or inducing the remission of) and/or preventing (acting as a prophylaxis) an inflammatory bowel disease or an inflammatory bowel disorder (both collectively referred to as IBD), Ulcerative Colitis; Crohn's disease; J-pouch; fistulising Crohn's disease; a Colitis which can be microscopic, lymphocytic or collagenous; an eosinophilic colitis; indeterminate colitis; idiopathic colitis; diverticulosis and diverticulitis; relapsing diverticulitis; constipation associated inflammatory bowel disease and/or small intestinal bacterial overgrowth; Irritable Bowel Syndrome (IBS) with or without diarrhoea, constipation or pain predominant IBS; periodontitis; rheumatoid arthritis; respiratory infections, appendicitis, vascular disorders such as thrombophlebitis; bacteremia; osteomyelitis; septic shock; Alzheimer's disease; Lemierre syndrome (postanginal sepsis); colonic polyps or adenomas (optionally hyperplastic, adenomatous or serrated adenomas) or preventing the growth of, or slowing the progression or recurrence of, colonic polyps or adenomas, bowl cancer, or metastases (optionally preventing the initiation of or recurrence of or promotion of bowl cancer or metastasis); pharyngitis; otitis; sinusitis; and any disease, symptom or condition caused or exacerbated by a Fusobacteria, e.g., a F. nucleatum or F. varium infection. In alternative embodiments, these pharmaceutical compositions, therapeutic combinations, devices and methods are custom dosaged and administered to both adults and children in need thereof. In alternative embodiments, pharmaceutical compositions or therapeutic combinations as provided herein are dosaged, formulated and/or administered as solid, gel, liquid or aerosol preparations or formulations. In alternative embodiments, pharmaceutical compositions or therapeutic combinations comprise rifaximin alone or in combination with other antibiotics or drugs.

### BACKGROUND

Inflammatory bowel diseases are numerous and include those caused by known infective agents such as Salmonella, Shigella, Campylobacter, Aeromonas, Clostridium difficile or Mycobacteria. Once the causative agents are eradicated by the endogenous microbiome or by specific therapy, the inflammatory process, which is visible as ‘colitis’ colonoscopically in such patients, resolves and the mucosa returns to being uninflamed. But essentially, there has to be an infective agent causing an inflammatory process to see a ‘colitis’. So detectable infective colitis is treated by treating the infection to eradicate the inflammation.

However, there is a group of patients, perhaps the largest segment of colitis, in which the infective agent cannot be identified. This “unidentified infective agent” group has variable symptoms and includes diagnoses such as idiopathic ulcerative colitis, Crohn's disease, lymphocytic colitis, collagenous colitis, microscopic colitis, diverticulitis with inflammation, and colitis caused by a drug when patients are treated for cancer (e.g., a ‘check point inhibitor’ gastrointestinal complication and may include symptoms as listed above). Another form of colitis is pouchitis, which is a common condition characterized by inflammation of the new rectum fashioned surgically to resemble a pouch in patients with chronic ulcerative colitis who have undergone total colectomy.

Although the gastrointestinal (GI) flora primarily include bacterial phyla that are non-pathogenic, the GI flora may also have pathogens such as Clostridia or Enterococci. When these pathogenic bacteria are present in the large intestine, even in healthy people, they are separated from the colonic wall by an impenetrable mucus layer, which is also called a biofilm. This biofilm layer is disturbed in IBD patients, thereby allowing bacteria to adhere to the exposed mucosa, which can also result in the invasion of epithelial cells by the bacteria, which may lead to the development of IBD. Additionally, in patients with IBD, the mucosal bacteria is present in a much higher concentration than in a healthy person and this concentration is proportional to the severity of the disease. This observation motivated use of antibiotics to induce remission in IBD; however, in spite of numerous clinical attempts, while the outcome for some antibiotics seems to cause an IBD remission, the majority of clinical trials had poor results, where placebo effects were barely separable from the antibiotic administration group (see, e.g., Gionchetti P., et al 1999; Perencevich, M., 2006). Trials with combinations of antibiotics including amoxicillin, tetracycline and metronidazole, while achieving a statistically significant suppression of IBD inflammation, did not cure the IBD condition (see e.g., Ohkusa, T et al 2005 and 2010).

### SUMMARY OF INVENTION

In a first aspect of the invention, there is provided a method for treating, ameliorating, reversing, causing the remission of, and/or preventing (acting as a prophylaxis, or preventing the initiation of) an inflammatory bowel disease or disorder (IBD); ulcerative colitis; Crohn's disease; J-pouch; fistulising Crohn's disease; a colitis which can be microscopic, lymphocytic or collagenous; an eosinophilic colitis; indeterminate colitis; idiopathic colitis; diverticulosis; diverticulitis; relapsing diverticulitis; constipation associated inflammatory bowel disease and/or small intestinal bacterial overgrowth; irritable bowel syndrome (IBS) with or without diarrhoea, constipation or pain predominant IBS; periodontitis; rheumatoid arthritis; respiratory infections, appendicitis, vascular disorders such as thrombophlebitis; bacteremia; osteomyelitis; septic shock; Alzheimer's disease; Lemierre syndrome (postanginal sepsis); colonic polyps; or adenomas (optionally hyperplastic, adenomatous or serrated adenomas); or preventing the growth of, or slowing the progression or recurrence of, colonic polyps or adenomas, bowl cancer, or metastases optionally preventing the initiation of or recurrence of or promotion of bowl cancer or metastasis; pharyngitis; otitis; or sinusitis; or treating, ameliorating, reversing, causing the remission of, and/or preventing any disease, symptom or condition caused or exacerbated by a Fusobacteria infection, e.g., a F. nucleatum, F. varium, F. simae, F. periodonticum, F. equimun, or F. necrogenes) infection, in an individual in need thereof, comprising administering to the individual in need thereof (optionally, a human or animal) a formulation, a pharmaceutical preparation, a therapeutic combination, or a pharmaceutical composition comprising or consisting of:

(a) (i) a rifaximin (or, In-25-yl acetate; (16Z,18E,28E)-5,6,21,23-tetrahydroxy-27-methoxy-2,4,11,16,20,22,24,26-octamethyl-1,15-dioxo-1,2-dihydro-2,7-(epoxypentadeca[1,11,13]trienoimino)furo[2″,3″:7′,8′]naphtho[1′,2′:4,5]imidazo[1,2-a]pyrid; or, 2S-Acetyloxy-5,6,21,23-tetrahydroxy-27-methoxy-2,4,11, 16,20,22,24,26-octamethyl-2,7-(epoxypentoeleca(1,11,13)trienimino)benzofuro[4,5-e]pyride[1,2-a]benzimidazole-1,15(2H)-dione; 80621-81-4; or, enantiomers or stereoisomers thereof) (optionally a XIFAXAN™, XIFAXANTA™, RITACOL™, FATROXIMIN™, XIFAXSAN™, RIFAXIMINUM™, RIFAXIMINUN™, RIFAXIMINE™, RIFAXIMIN™, RIFAXIDIN™, RIFAXIMINA™, RIFAMYCIN™, VETRANAL™, or NORMIX™), a polymorphic form of a rifaximin or a rifaximin equivalent thereof, an extended intestinal release (EIR) rifaximin, a rifamycin derivative, a rifampicin (or rifampin) (optionally RIFADIN™), a rifabutin (optionally MYCOBUTIN™), a rifapentin (optionally PRIFTIN™), a rifalazil, a bicozamycin, a pyrido-imidazo rifamycin, a dehydro-rifaximin, a rifaximin histidine, a rifaximin tryptophan, an 11-desmethyl-rifaximin (e.g., an 11-desmethyl NORMIX™), a rifaximin beta-cyclodextrin, fosfomycin, or equivalents thereof or a mixture or a combination thereof,
(ii) vancomycin, neomycin, tobramycin, paromomycin or streptomycin,
(iii) gentamicin, streptomycin, ridinilazole, teicoplanin, streptomycin and neomycin, fidaxomicin, ramoplanin, surotomycin, capozide, a partially absorbed agent, optionally comprising a tinidazole, metronidazole, nitazoxanide, amoxicillin, tetracyclines, ornidazole, secnidazole, ciprofloxacin or an ansamycin, or
(iv) an antibiotic or drug as listed in Table 1; or
(b) an antibiotic or drug of (a), and at least one additional antimicrobial or antibiotic agent, wherein optionally for (a) or (b) the rifaximin or rifaximin polymorphic form thereof or rifaximin equivalent comprises:
(i) a rifaximin, a rifaximin polymorph or a rifaximin equivalent as described in U.S. Pat. No. 9,273,066, optionally comprising a polymorphic form zeta of rifaximin exhibiting an X-ray powder diffraction pattern having characteristic peaks expressed in degrees 2 theta (+/−0.20 degree theta) comprising 4.69, 7.63, 12.52, 13.87;
(ii) a 25-desacetyl rifaximin, or a rifaximin, a rifaximin polymorph or a rifaximin equivalent, as described in U.S. Pat. No. 9,364,467, optionally comprising a 25-desacetyl rifaximin or a pharmaceutically acceptable salt thereof (optionally a sodium, potassium, calcium, magnesium, ammonium, or chlorine pharmaceutically acceptable salt of 25-desacetyl rifaximin), wherein optionally the 25-desacetyl rifaximin has the formula:

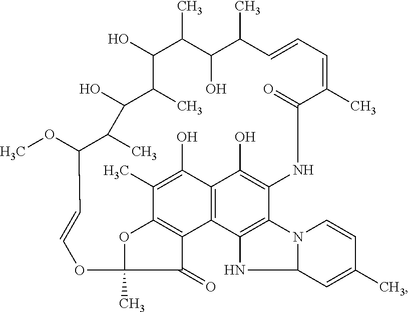 [SMILES: COC1/C=C/O[C@@]2(C)Oc3c(C)c(O)c4c(O)c(c5c(c4c3C2=O)NC2C=C(C)C=CN52)NC(=O)/C(C)=C\C=C\C(C)C(O)C(C)C(O)C(C)C(O)C1C]

(iii) a rifaximin, a rifaximin polymorph or a rifaximin equivalent as described in U.S. Pat. No. 9,546,183, optionally comprising a polymorphic Form B of rifaximin exhibiting an X-ray powder diffraction pattern having characteristic peaks expressed in degrees 2 theta (+/−0.20 degree theta) at 5.24, 6.84, 7.74, 8.71, 10.16, and 12.21,
(iv) a rifaximin amorphous form or a rifaximin equivalent as described in U.S. Pat. No. 9,700,545, optionally comprising an amorphous form of rifaximin exhibiting an X-ray powder diffraction pattern having characteristic peaks expressed in degrees: (1) 2 theta (+/−0.20 degree theta) at 7.3, 11.3-17.8, and 15.8 degrees 2 theta; 2 theta (+/−0.20 degree theta) at 5.1-10.1, 11.3-17.8, and 15.8 degrees 2 theta; or (3) 2 theta (+/−0.20 degree theta) at 5.1-10.1, 7.3, and 11.3-17.8 degrees 2 theta,
(v) a rifaximin, a rifaximin polymorph or a rifaximin equivalent as described in U.S. Pat. No. 9,359,374, or U.S. Pat. No. 9,725,466, optionally comprising a polymorphic form APO-III of rifaximin characterized by a PXRD diffractogram comprising peaks, in terms of degrees 2-theta, at approximately 7.1, 8.4, 11.6, 13.1, 18.5, 18.8, and 25.0,
(vi) a rifaximin, a rifaximin polymorph or a rifaximin equivalent as described in U.S. Pat. No. 9,421,195,
(vii) a rifaximin, a rifaximin polymorph or a rifaximin equivalent as described in U.S. Pat. No. 7,045,620, optionally a crystalline polymorphous form of a rifaximin, a rifaximin polymorph or a rifaximin equivalent; and/or
(viii) a controlled-release or spray-dried rifaximin, rifaximin polymorph or rifaximin equivalent as described in U.S. Pat. No. 9,498,442, optionally rifaximin, rifaximin polymorph or rifaximin equivalent characterized by an X-Ray diffraction spectrum showing diffraction halo peaks in the range 7.75 degree+−0.0.2-18.33 degree±0.0.2, 2 theta, with maximum at about 7.75 degree±0.0.2 and in the range 14.54 degree±0.0.2 and 18.33 degree±0.0.2, 2 theta.

In a second aspect of the invention, there is provided a use of a pharmaceutical composition comprising or consisting of:

(a) (i) a rifaximin (or, In-25-yl acetate; (16Z,18E,28E)-5,6,21,23-tetrahydroxy-27-methoxy-2,4,11,16,20,22,24,26-octamethyl-1,15-dioxo-1,2-dihydro-2,7-(epoxypentadeca[1,11,13]trienoimino)furo[2″,3″:7′,8′]naphtho[1′,2′:4,5]imidazo[1,2-a]pyrid; or, 2S-Acetyloxy-5,6,21,23-tetrahydroxy-27-methoxy-2,4,11, 16,20,22,24,26-octamethyl-2,7-(epoxypentoeleca(1,11,13)trienimino)benzofuro[4,5-e]pyride[1,2-a]benzimidazole-1,15(2H)-dione; 80621-81-4; or, enantiomers or stereoisomers thereof) (optionally a XIFAXAN™, XIFAXANTA™, RITACOL™, FATROXIMIN™, XIFAXSAN™, RIFAXIMINUM™, RIFAXIMINUN™, RIFAXIMINE™, RIFAXIMIN™, RIFAXIDIN™, RIFAXIMINA™, RIFAMYCIN™, VETRANAL™, or NORMIX™), a polymorphic form of a rifaximin or a rifaximin equivalent thereof, an extended intestinal release (EIR) rifaximin, a rifamycin derivative, a rifampicin (or rifampin) (optionally RIFADIN™), a rifabutin (optionally MYCOBUTIN™), a rifapentin (optionally PRIFTIN™), a rifalazil, a bicozamycin, a pyrido-imidazo rifamycin, a dehydro-rifaximin, a rifaximin histidine, a rifaximin tryptophan, an 11-desmethyl-rifaximin (e.g., an 11-desmethyl NORMIX™), a rifaximin beta-cyclodextrin, fosfomycin, or equivalents thereof or a mixture or a combination thereof,
(ii) vancomycin, neomycin, tobramycin, paromomycin or streptomycin,
(iii) gentamicin, streptomycin, ridinilazole, teicoplanin, streptomycin and neomycin, fidaxomicin, ramoplanin, surotomycin, capozide, a partially absorbed agent, optionally comprising a tinidazole, metronidazole, nitazoxanide, amoxicillin, tetracyclines, ornidazole, secnidazole, ciprofloxacin or an ansamycin, or
(iv) an antibiotic or drug as listed in Table 1; or
(b) an antibiotic or drug of (a), and at least one additional antimicrobial or antibiotic agent, wherein optionally for (a) or (b) the rifaximin or rifaximin polymorphic form thereof or rifaximin equivalent comprises:
(i) a rifaximin, a rifaximin polymorph or a rifaximin equivalent as described in U.S. Pat. No. 9,273,066, optionally comprising a polymorphic form zeta of rifaximin exhibiting an X-ray powder diffraction pattern having characteristic peaks expressed in degrees 2 theta (+/−0.20 degree theta) comprising 4.69, 7.63, 12.52, 13.87;
(ii) a 25-desacetyl rifaximin, or a rifaximin, a rifaximin polymorph or a rifaximin equivalent, as described in U.S. Pat. No. 9,364,467, optionally comprising a 25-desacetyl rifaximin or a pharmaceutically acceptable salt thereof (optionally a sodium, potassium, calcium, magnesium, ammonium, or chlorine pharmaceutically acceptable salt of 25-desacetyl rifaximin), wherein optionally the 25-desacetyl rifaximin has the formula:

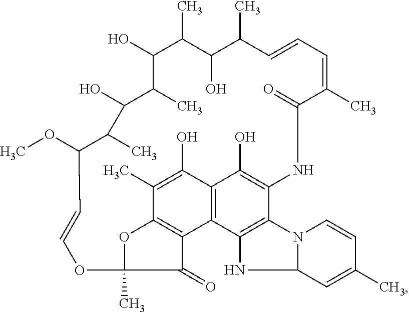 [SMILES: COC1/C=C/O[C@@]2(C)Oc3c(C)c(O)c4c(O)c(c5c(c4c3C2=O)NC2C=C(C)C=CN52)NC(=O)/C(C)=C\C=C\C(C)C(O)C(C)C(O)C(C)C(O)C1C]

(iii) a rifaximin, a rifaximin polymorph or a rifaximin equivalent as described in U.S. Pat. No. 9,546,183, optionally comprising a polymorphic Form B of rifaximin exhibiting an X-ray powder diffraction pattern having characteristic peaks expressed in degrees 2 theta (+/−0.20 degree theta) at 5.24, 6.84, 7.74, 8.71, 10.16, and 12.21,
(iv) a rifaximin amorphous form or a rifaximin equivalent as described in U.S. Pat. No. 9,700,545, optionally comprising an amorphous form of rifaximin exhibiting an X-ray powder diffraction pattern having characteristic peaks expressed in degrees: (1) 2 theta (+/−0.20 degree theta) at 7.3, 11.3-17.8, and 15.8 degrees 2 theta; 2 theta (+/−0.20 degree theta) at 5.1-10.1, 11.3-17.8, and 15.8 degrees 2 theta; or (3) 2 theta (+/−0.20 degree theta) at 5.1-10.1, 7.3, and 11.3-17.8 degrees 2 theta,
(v) a rifaximin, a rifaximin polymorph or a rifaximin equivalent as described in U.S. Pat. No. 9,359,374, or U.S. Pat. No. 9,725,466, optionally comprising a polymorphic form APO-III of rifaximin characterized by a PXRD diffractogram comprising peaks, in terms of degrees 2-theta, at approximately 7.1, 8.4, 11.6, 13.1, 18.5, 18.8, and 25.0,
(vi) a rifaximin, a rifaximin polymorph or a rifaximin equivalent as described in U.S. Pat. No. 9,421,195,
(vii) a rifaximin, a rifaximin polymorph or a rifaximin equivalent as described in U.S. Pat. No. 7,045,620, optionally a crystalline polymorphous form of a rifaximin, a rifaximin polymorph or a rifaximin equivalent; and/or
(viii) a controlled-release or spray-dried rifaximin, rifaximin polymorph or rifaximin equivalent as described in U.S. Pat. No. 9,498,442, optionally rifaximin, rifaximin polymorph or rifaximin equivalent characterized by an X-Ray diffraction spectrum showing diffraction halo peaks in the range 7.75 degree+−0.0.2-18.33 degree±0.0.2, 2 theta, with maximum at about 7.75 degree±0.0.2 and in the range 14.54 degree±0.0.2 and 18.33 degree±0.0.2, 2 theta, in the manufacture of a medicament for treating, ameliorating, reversing, causing the remission of, and/or preventing (acting as a prophylaxis, or preventing the initiation of) an inflammatory bowel disease or disorder (IBD); ulcerative colitis; Crohn's disease; J-pouch; fistulising Crohn's disease; a colitis which can be microscopic, lymphocytic or collagenous; an eosinophilic colitis; indeterminate colitis; idiopathic colitis; diverticulosis; diverticulitis; relapsing diverticulitis; constipation associated inflammatory bowel disease and/or small intestinal bacterial overgrowth; irritable bowel syndrome (IBS) with or without diarrhoea, constipation or pain predominant IBS; periodontitis; rheumatoid arthritis; respiratory infections, appendicitis, vascular disorders such as thrombophlebitis; bacteremia; osteomyelitis; septic shock; Alzheimer's disease; Lemierre syndrome (postanginal sepsis); colonic polyps; or adenomas (optionally hyperplastic, adenomatous or serrated adenomas); or
preventing the growth of, or slowing the progression or recurrence of, colonic polyps or adenomas, bowl cancer, or metastases optionally preventing the initiation of or recurrence of or promotion of bowl cancer or metastasis; pharyngitis; otitis; or sinusitis; or
treating, ameliorating, reversing, causing the remission of, and/or preventing any disease, symptom or condition caused or exacerbated by a Fusobacteria infection, e.g., a F. nucleatum, F. varium, F. simae, F. periodonticum, F. equimun, or F. Necrogenes) infection,
in an individual in need thereof.

In a third aspect of the invention, there is provided a pharmaceutical composition for use in treating, ameliorating, reversing, causing the remission of, and/or preventing (acting as a prophylaxis, or preventing the initiation of) an inflammatory bowel disease or disorder (IBD); ulcerative colitis; Crohn's disease; J-pouch; fistulising Crohn's disease; a colitis which can be microscopic, lymphocytic or collagenous; an eosinophilic colitis; indeterminate colitis; idiopathic colitis; diverticulosis; diverticulitis; relapsing diverticulitis; constipation associated inflammatory bowel disease and/or small intestinal bacterial overgrowth; irritable bowel syndrome (IBS) with or without diarrhoea, constipation or pain predominant IBS; periodontitis; rheumatoid arthritis; respiratory infections, appendicitis, vascular disorders such as thrombophlebitis; bacteremia; osteomyelitis; septic shock; Alzheimer's disease; Lemierre syndrome (postanginal sepsis); colonic polyps; or adenomas (optionally hyperplastic, adenomatous or serrated adenomas); or

preventing the growth of, or slowing the progression or recurrence of, colonic polyps or adenomas, bowl cancer, or metastases optionally preventing the initiation of or recurrence of or promotion of bowl cancer or metastasis; pharyngitis; otitis; or sinusitis; or
treating, ameliorating, reversing, causing the remission of, and/or preventing any disease, symptom or condition caused or exacerbated by a Fusobacteria infection, e.g., a F. nucleatum, F. varium, F. simae, F. periodonticum, F. equimun, or F. Necrogenes) infection,
in an individual in need thereof, the pharmaceutical composition comprising or consisting of:
(a) (i) a rifaximin (or, In-25-yl acetate; (16Z,18E,28E)-5,6,21,23-tetrahydroxy-27-methoxy-2,4,11,16,20,22,24,26-octamethyl-1,15-dioxo-1,2-dihydro-2,7-(epoxypentadeca[1,11,13]trienoimino)furo[2″,3″:7′,8′]naphtho[1′,2′:4,5]imidazo[1,2-a]pyrid; or, 2S-Acetyloxy-5,6,21,23-tetrahydroxy-27-methoxy-2,4,11, 16,20,22,24,26-octamethyl-2,7-(epoxypentoeleca(1,11,13)trienimino)benzofuro[4,5-e]pyride[1,2-a]benzimidazole-1,15(2H)-dione; 80621-81-4; or, enantiomers or stereoisomers thereof) (optionally a XIFAXAN™, XIFAXANTA™, RITACOL™, FATROXIMIN™, XIFAXSAN™, RIFAXIMINUM™, RIFAXIMINUN™, RIFAXIMINE™, RIFAXIMIN™, RIFAXIDIN™, RIFAXIMINA™, RIFAMYCIN™, VETRANAL™, or NORMIX™), a polymorphic form of a rifaximin or a rifaximin equivalent thereof, an extended intestinal release (EIR) rifaximin, a rifamycin derivative, a rifampicin (or rifampin) (optionally RIFADIN™), a rifabutin (optionally MYCOBUTIN™), a rifapentin (optionally PRIFTIN™), a rifalazil, a bicozamycin, a pyrido-imidazo rifamycin, a dehydro-rifaximin, a rifaximin histidine, a rifaximin tryptophan, an 11-desmethyl-rifaximin (e.g., an 11-desmethyl NORMIX™), a rifaximin beta-cyclodextrin, fosfomycin, or equivalents thereof or a mixture or a combination thereof,
(ii) vancomycin, neomycin, tobramycin, paromomycin or streptomycin,
(iii) gentamicin, streptomycin, ridinilazole, teicoplanin, streptomycin and neomycin, fidaxomicin, ramoplanin, surotomycin, capozide, a partially absorbed agent, optionally comprising a tinidazole, metronidazole, nitazoxanide, amoxicillin, tetracyclines, ornidazole, secnidazole, ciprofloxacin or an ansamycin, or
(iv) an antibiotic or drug as listed in Table 1; or
(b) an antibiotic or drug of (a), and at least one additional antimicrobial or antibiotic agent,
wherein optionally for (a) or (b) the rifaximin or rifaximin polymorphic form thereof or rifaximin equivalent comprises:
(i) a rifaximin, a rifaximin polymorph or a rifaximin equivalent as described in U.S. Pat. No. 9,273,066, optionally comprising a polymorphic form zeta of rifaximin exhibiting an X-ray powder diffraction pattern having characteristic peaks expressed in degrees 2 theta (+/−0.20 degree theta) comprising 4.69, 7.63, 12.52, 13.87;
(ii) a 25-desacetyl rifaximin, or a rifaximin, a rifaximin polymorph or a rifaximin equivalent, as described in U.S. Pat. No. 9,364,467, optionally comprising a 25-desacetyl rifaximin or a pharmaceutically acceptable salt thereof (optionally a sodium, potassium, calcium, magnesium, ammonium, or chlorine pharmaceutically acceptable salt of 25-desacetyl rifaximin), wherein optionally the 25-desacetyl rifaximin has the formula:

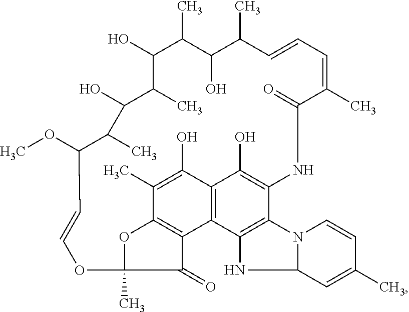 [SMILES: COC1/C=C/O[C@@]2(C)Oc3c(C)c(O)c4c(O)c(c5c(c4c3C2=O)NC2C=C(C)C=CN52)NC(=O)/C(C)=C\C=C\C(C)C(O)C(C)C(O)C(C)C(O)C1C]

(iii) a rifaximin, a rifaximin polymorph or a rifaximin equivalent as described in U.S. Pat. No. 9,546,183, optionally comprising a polymorphic Form B of rifaximin exhibiting an X-ray powder diffraction pattern having characteristic peaks expressed in degrees 2 theta (+/−0.20 degree theta) at 5.24, 6.84, 7.74, 8.71, 10.16, and 12.21,
(iv) a rifaximin amorphous form or a rifaximin equivalent as described in U.S. Pat. No. 9,700,545, optionally comprising an amorphous form of rifaximin exhibiting an X-ray powder diffraction pattern having characteristic peaks expressed in degrees: (1) 2 theta (+/−0.20 degree theta) at 7.3, 11.3-17.8, and 15.8 degrees 2 theta; 2 theta (+/−0.20 degree theta) at 5.1-10.1, 11.3-17.8, and 15.8 degrees 2 theta; or (3) 2 theta (+/−0.20 degree theta) at 5.1-10.1, 7.3, and 11.3-17.8 degrees 2 theta,
(v) a rifaximin, a rifaximin polymorph or a rifaximin equivalent as described in U.S. Pat. No. 9,359,374, or U.S. Pat. No. 9,725,466, optionally comprising a polymorphic form APO-III of rifaximin characterized by a PXRD diffractogram comprising peaks, in terms of degrees 2-theta, at approximately 7.1, 8.4, 11.6, 13.1, 18.5, 18.8, and 25.0,
(vi) a rifaximin, a rifaximin polymorph or a rifaximin equivalent as described in U.S. Pat. No. 9,421,195,
(vii) a rifaximin, a rifaximin polymorph or a rifaximin equivalent as described in U.S. Pat. No. 7,045,620, optionally a crystalline polymorphous form of a rifaximin, a rifaximin polymorph or a rifaximin equivalent; and/or
(viii) a controlled-release or spray-dried rifaximin, rifaximin polymorph or rifaximin equivalent as described in U.S. Pat. No. 9,498,442, optionally rifaximin, rifaximin polymorph or rifaximin equivalent characterized by an X-Ray diffraction spectrum showing diffraction halo peaks in the range 7.75 degree+−0.0.2-18.33 degree±0.0.2, 2 theta, with maximum at about 7.75 degree±0.0.2 and in the range 14.54 degree±0.0.2 and 18.33 degree±0.0.2, 2 theta.

The following are embodiments which may be combined (alone or in any combination) with the methods, uses and pharmaceutical compositions for use of the first to third aspects of the invention, as provided above.

The IBD further comprises or is associated with a condition or side effect comprising diarrhoea, rectal bleeding, mucus, urgency, incontinence, nocturnal diarrhoea; lower abdominal pain, weight loss, excessive gas production, bloating, loss of appetite, joint pains/symptoms, and optionally administration of the formulation, pharmaceutical preparation, therapeutic combination or pharmaceutical composition treats, ameliorates, reverses, causes the remission of, and/or prevents (acts as a prophylaxis) one, several or all of these conditions or side effects.

The formulation, pharmaceutical preparation, therapeutic combination or pharmaceutical composition further comprises at least one additional antimicrobial or antibiotic agent, or further comprises a drug or a probiotic.

The at least one additional antimicrobial or antibiotic agent comprises a vancomycin, a metronidazole (optionally FLAGYL™, METRO™), a tinidazole (optionally FASIGYN™ SIMPLOTAN™, TINDAMAX™), an ornidazole (optionally XYNOR™), a secnidazole (optionally FLAGENTYL™, SINDOSE™, SECNIL™), an antibiotic or drug as listed in Table 1, or a combination thereof.

The at least one additional antimicrobial or antibiotic agent comprises: an antibiotic or antibacterial agent from one or more of the following classes selected from: tetracyclines, penicillins, macrolides, quinolones, chloramphenicol, rifamycins, sulphonamides, co-trimoxazole, and oxazolidinones.

The at least one additional antimicrobial or antibiotic agent comprises: a doxycycline, chlortetracycline, tetracycline hydrochloride, oxytetracycline, demeclocycline, methacycline, minocycline, penicillin, amoxycillin, erythromycin, clarithromycin, roxithromycin, azithromycin, spiramycin, oleandomycin, josamycin, kitsamysin, flurithromycin, nalidixic acid, oxolinic acid, norfloxacin, perfloxacin, amifloxacin, ofloxacin, moxifloxacin, ciprofloxacin, sparfloxacin, levofloxacin, rifabutin, rifampicin, rifapentin, rifalazil, sulfisoxazole, sulfamethoxazole, sulfadiazine, sulfadoxine, sulfasalazine, sulfaphenazole, dapsone, sulfacytidine, linezolid or any combination thereof.

The at least one additional antimicrobial or antibiotic agent comprises:

an ampicillin, a sulbactama tetracycline, a cephalosporin, a carbapenem, an imipenem, a meropenem, a monobactam, a lincosamide, a clindamycin, a quinolone, a fluoroquinolone, a sulphonamide, a fradicin, a nitroimidazole, a metronidazole, a tinidazole, a secnidazole, an anti-Clostridial agent, or a ramoplanan,
an aminoglycoside antibiotic, a gentamycin, a neomycin, a streptomycin, a paromomycin, a verdamicin, a mutamicin, a sisomicin, a netilmicin, a retymicin, a kanamycin, an amphenicol, an ansamycin, a beta-lactam (β-lactam) antibiotic, a carbapenem, a cephalosporin, a cephamycin, a monobactam, an oxacephem, a lincosamide antibiotic, a clindamycin, or a lincomycin, a glycopeptide antibiotic, a vancomycin, a teicoplanin, a telavancin, a bleomycin, a ramoplanin, a decaplanin, a polypeptide antibiotic, an actinomycin, an actinomycin D, a bacitracin, a bacitracin, a tetracycline, a 2,4-diaminopyrimidine class antibiotic, a clavacin, a clairformin, a claviform, an expansine, a clavatin, an expansin, a gigantin, a leucopin, a patuline or a patulin, or
an equivalent thereof or a combination thereof.

The at least one additional antimicrobial or antibiotic agent comprises:

(i) a rifaximin (optionally a XIFAXAN™, XIFAXANTA™, RITACOL™, FATROXIMIN™, XIFAXSAN™, RIFAXIMINUM™, RIFAXIMINUN™, RIFAXIMINE™, RIFAXIMIN™, RIFAXIDIN™, RIFAXIMINA™, RIFAMYCIN™, or NORMIX™), a polymorphic form of a rifaximin or a rifaximin equivalent thereof, an extended intestinal release (EIR) rifaximin, a rifamycin derivative, a rifampicin (or rifampin) (optionally RIFADIN™), a rifabutin (optionally MYCOBUTIN™), a rifapentin (optionally PRIFTIN™), a rifalazil, a bicozamycin, a pyrido-imidazo rifamycin, a dehydro-rifaximin, a rifaximin histidine, a rifaximin tryptophan, an 11-desmethyl-rifaximin (e.g., an 11-desmethyl NORMIX™), a rifaximin beta-cyclodextrin, or equivalents thereof or a mixture or a combination thereof,
(ii) vancomycin, neomycin, tobramycin, paromomycin or streptomycin,
(iii) gentamicin, streptomycin, ridinilazole, teicoplanin, streptomycin and neomycin, fidaxomicin, ramoplanin, surotomycin, capozide, a partially absorbed agent, optionally comprising a tinidazole, metronidazole, nitazoxanide, amoxicillin, tetracyclines, ornidazole, secnidazole, ciprofloxacin or an ansamycin, or
(iv) an antibiotic or drug as listed in Table 1, or
(v) any combination thereof.

The formulation, pharmaceutical preparation, therapeutic combination, or pharmaceutical composition comprises a rifamycin, a nitroimidazole, and a tetracycline antibiotic. In some embodiments, the rifamycin is rifampicin, the nitroimidazole is secnidazole, and the tetracycline antibiotic is doxycycline.

The formulation, pharmaceutical preparation, therapeutic combination, or pharmaceutical composition comprises a rifamycin, a nitroimidazole, and a thiazolide. In some embodiments, the rifamycin is rifaximin, the nitroimidazole is tinidazole, and the thiazolide is nitazoxanide.

The formulation, pharmaceutical preparation, therapeutic combination, or pharmaceutical composition comprises fosfomycin, a nitroimidazole, and a tetracycline antibiotic. In some embodiments, the nitroimidazole is metronidazole, and the tetracycline antibiotic is doxycycline.

The antimicrobial or antibiotic agent comprises a three-drug therapeutic combination comprising: rifaximin, tinidazole and nitazoxanide; rifamycin, secnidazole, and doxycycline; rifaximin, tinidazole, and nitazoxanide; rifaximin, tinidazole and oral tobramycin; rifaximin, amoxicillin and metronidazole; rifaximin, paromomycin and nitazoxanide; rifaximin, paromomycin and vancomycin; rifaximin, tinidazole and paromomycin; rifaximin, ridinilazole and tobramycin; rifaximin, ridinilazole and paromomycin; or rifaximin, ridinilazole and nitazoxanide; and optionally teicoplanin is substituted for any one of the second or third drug in this 3-drug combination list.

The individual exhibits at least an about 5% to 10%, or 10% to 20%, or 20% to 30%, or 30% to 40%, or 40% to 50%, or 50% to 60%, or 70% to 80%, or 80% to 90%, or substantially complete, reduction in IBD symptoms or side effects or severity after administration of the formulation, pharmaceutical preparation, therapeutic combination or pharmaceutical composition to the individual in need thereof as compared to before initiating the administration.

The at least an about 5% to 10%, or 10% to 20%, or 20% to 30%, or 30% to 40%, or 40% to 50%, or 50% to 60%, or 70% to 80%, or 80% to 90%, or substantially complete, reduction in IBD symptoms or side effects or severity is achieved after about 1, 2 or 3 or more weeks, or after about 1 to 2 months, or after about 2 to 6 months, of initiating the administration.

The at least an about 5% to 10%, or 10% to 20%, or 20% to 30%, or 30% to 40%, or 40% to 50%, or 50% to 60%, or 70% to 80%, or 80% to 90%, or substantially complete, reduction in IBD symptoms or side effects or severity is maintained for at least about 4 to 8 weeks, or 2 to 6 months, after discontinuing the administration to the individual.

The formulation, pharmaceutical preparation, therapeutic combination or pharmaceutical composition is formulated as a chewable delivery vehicle, a gum, a gummy, a candy, a lozenge, an ice cream or an ice, a yogurt or a drink.

A unit dosage of the formulation, pharmaceutical preparation, therapeutic combination or pharmaceutical composition is a pediatric unit dosage, and optionally the unit dosage is between about 10 mg and 1100 mgm, or between about between about 40 mg and 4,000 mgm, or is about 10, 20, 30, 40, 50, 60, 70, 75, 80, 90, 100, 125, 150, 175, 200, 225, 250, 275, 300, 325, 350, 375, 400, 425, 450, 475, 500, 600, 700, 750, 800, 900, 1000, 1100, 1500, 2000, 2500, 3000, 3500, 4000, or more mg per unit dose, which optionally can be administered once a day, bid or tid, or a four times a day, five times a day or six times a day or more, regimen.

A daily dosage of the formulation, pharmaceutical preparation, therapeutic combination or pharmaceutical composition is about 50, 75, 100, 125, 150, 175, 200, 225, 250, 275, 300, 325, 350, 375, 400, 425, 450, 475, 500, 600, 700, 750, 800, 900, 1000, 1100, 1500, 2000, 2500, 3000, 3500, 4000, or more mg per day, or between about 100 and 1100 mgm per day total, or between about 400 and 4000 mg per day, which optionally can be administered in a once a day, bid or tid, or four times a day, five times a day or six times a day or more, regimen.

A unit dosage of the formulation, pharmaceutical preparation, therapeutic combination or pharmaceutical composition is set for (the daily dosage is set for) bid (twice a day), tid (three times a day), four times a day, five times a day or six times a day or more, with the unit dosage and daily dosage adjusted to be: about 1000 mg/70 kg a day, or about 14 mg/kg a day, for an adult median dose per day; or for a pediatric dosage about 350 mg/25 kg a day, or about 15 to 16 mg/kg, a day; or equivalent.

The daily dosage of the formulation, pharmaceutical preparation, therapeutic combination or pharmaceutical composition is about 25 mg to 20 grams (gm) bid, or about 400, 425, 450, 475, 500, 600, 700, 750, 800, 900, 1000, 1100, 1200, 1500, 2000, 2500, 3000, 3500, 4000, or more mgm once a day, bid or tid, or four times a day, five times a day or six times a day or more.

The daily dosage of the formulation, pharmaceutical preparation, therapeutic combination or pharmaceutical composition, or one ingredient of the formulation, pharmaceutical preparation, therapeutic combination or pharmaceutical composition, is increased or “ramped up” every week, or every other week, by about 25, 50, 75, 100, 125, 150, 175, 200, 225, 250, 275, 300, 325, 350, 375, 400, 425, 450, 475, 1500, 2000, 2500, 3000, 3500, 4000, or more or more mg per week, or every other week, and optionally this “ramping up” or increasing of dosages continues for about a month, about 6 months or about a year, or until symptoms of IBD significantly diminish or abate, or significantly diminish or abate without need for administration of the formulation, the pharmaceutical or the pharmaceutical preparation.

The formulation, the pharmaceutical or the pharmaceutical preparation further comprises a flavoring or a sweetening agent, an aspartamine, a stevia, monk fruit, a sucralose, a saccharin, a cyclamate, a xylitol, a vanilla, an artificial vanilla or chocolate or strawberry flavor, an artificial chocolate essence, or a mixture or combination thereof.

The formulation, pharmaceutical preparation, therapeutic combination or pharmaceutical composition further comprises a preservative, a benzoic acid or a potassium sorbate.

The formulation, pharmaceutical preparation, therapeutic combination or pharmaceutical composition further comprises, or has added to: at least one probiotic or prebiotic, wherein optionally the prebiotic comprises an inulin, lactulose, extracts of artichoke, chicory root, oats, barley, various legumes, garlic, kale, beans or flacks or an herb, and optionally the probiotic comprises a cultured or stool-extracted microorganism or bacteria, or a bacterial component, and optionally the probiotic bacteria or bacterial component comprises or is derived from a non-pathogenic Clostridia, a Bacteroidetes, a Firmicutes, a Lactobacilli, a Bifidobacteria, an E. coli, a Strep fecalis, an Actinobacteria, a Proteobacteria, a Verruco-microbia, a Fusobacteria, a Cyanobacteria, a Spirochetes and a Lentisphaerae, and equivalents.

The formulation, pharmaceutical preparation, therapeutic combination or pharmaceutical composition further comprises, or has added to: at least one congealing agent, wherein optionally the congealing agent comprises an arrowroot or a plant starch, a powdered flour, a powdered potato or potato starch, an absorbant polymer, an Absorbable Modified Polymer, and/or a corn flour or a corn starch.

The formulation, pharmaceutical preparation, therapeutic combination or pharmaceutical composition further comprises an additive selected from one or more of a saline, a media, a defoaming agent, a surfactant agent, a lubricant, an acid neutralizer, a marker, a cell marker, a drug, an antibiotic, a contrast agent, a dispersal agent, a buffer or a buffering agent, a sweetening agent, a debittering agent, a flavoring agent, a pH stabilizer, an acidifying agent, a preservative, a desweetening agent and/or coloring agent, vitamin, mineral and/or dietary supplement, or a prebiotic nutrient.

The formulation, pharmaceutical preparation, therapeutic combination or pharmaceutical composition further comprises, or has added to: at least one Biofilm Disrupting Compound, wherein optionally the biofilm disrupting compound comprises an enzyme, a deoxyribonuclease (DNase), N-acetylcysteine, an auranofin, an alginate lyase, glycoside hydrolase dispersin B; a Quorum-sensing inhibitor, a ribonucleic acid III inhibiting peptide, Salvadora persica extracts, Competence-stimulating peptide, Patulin and penicillic acid; peptides—cathelicidin-derived peptides, small lytic peptide, PTP-7, Nitric oxide, neo-emulsions; ozone, lytic bacteriophages, lactoferrin, xylitol hydrogel, synthetic iron chelators, cranberry components, curcumin, silver nanoparticles, Acetyl-11-keto-β-boswellic acid (AKBA), barley coffee components, probiotics, sinefungin, S-adenosylmethionine, S-adenosyl-homocysteine, Delisea furanones, N-sulfonyl homoserine lactones or any combination thereof.

The formulation, pharmaceutical preparation, therapeutic combination or pharmaceutical composition is formulated as a delayed or gradual enteric release composition or formulation, and optionally the formulation comprises a gastro-resistant coating designed to dissolve at a pH of 7 in the terminal ileum, e.g., an active ingredient is coated with an acrylic based resin or equivalent, e.g., a poly(meth)acrylate, e.g. a methacrylic acid copolymer B, NF, which dissolves at pH 7 or greater, e.g., comprises a multimatrix (MMX) formulation.

The formulation, pharmaceutical preparation, therapeutic combination or pharmaceutical composition is contained in a delivery vehicle, product of manufacture, container, syringe, device or bag.t

The formulation, pharmaceutical preparation, therapeutic combination or pharmaceutical composition is initially manufactured or formulated as a liquid, a suspension, a gel, a geltab, a semisolid, a tablet, a sachet, a lozenge or a capsule, or as an enteral formulation, or re-formulated for final delivery as a liquid, a suspension, a gel, a geltab, a semisolid, a tablet, a sachet, a lozenge or a capsule, or as an enteral formulation.

In alternative embodiments, provided are products of manufacture comprising or having contained therein a formulation, pharmaceutical preparation, therapeutic combination or pharmaceutical composition as used in any method as provided herein, wherein optionally the product of manufacture is an implant or a kit.

The details of one or more embodiments of the invention are set forth in the accompanying description below. Other features, objects, and advantages of the invention will be apparent from the description and the claims.

All publications, patents, patent applications cited herein are hereby expressly incorporated by reference for all purposes.

In one aspect, forms of the invention include the following.

1. A method for treating, ameliorating, reversing, causing the remission of, and/or preventing (acting as a prophylaxis, or preventing the initiation of) an inflammatory bowel disease or disorder (IBD) or inflammatory bowel disease (IBD); Ulcerative ulcerative Colitiscolitis; Crohn's disease; J-pouch; fistulising Crohn's disease; a Colitis colitis which can be microscopic, lymphocytic or collagenous; an eosinophilic colitis; indeterminate colitis; idiopathic colitis; diverticulosis; and diverticulitis; relapsing diverticulitis; constipation associated inflammatory bowel disease and/or small intestinal bacterial overgrowth; Irritable irritable Bowel bowel Syndrome syndrome (IBS) with or without diarrhoea, constipation or pain predominant IBS; periodontitis; rheumatoid arthritis; respiratory infections, appendicitis, vascular disorders such as thrombophlebitis; bacteremia; osteomyelitis; septic shock; Alzheimer's disease; Lemierre syndrome (postanginal sepsis); colonic polyps; or adenomas (optionally hyperplastic, adenomatous or serrated adenomas); or
preventing the growth of, or slowing the progression or recurrence of, colonic polyps or adenomas, bowl cancer, or metastases optionally preventing the initiation of or recurrence of or promotion of bowl cancer or metastasis; pharyngitis; otitis; or sinusitis; and or
treating, ameliorating, reversing, causing the remission of, and/or preventing any disease, symptom or condition caused or exacerbated by a Fusobacteria infection, e.g., a F. nucleatum, F. varium, F. simae, F. periodonticum, F. equimun, or F. Necrogenes) infection,
in an individual in need thereof, comprising administering to the individual in need thereof (optionally, a human or animal) a formulation, a pharmaceutical preparation, a therapeutic combination, or a pharmaceutical composition comprising or consisting of:
(a) (i) a rifaximin (or, In-25-yl acetate; (16Z,18E,28E)-5,6,21,23-tetrahydroxy-27-methoxy-2,4,11,16,20,22,24,26-octamethyl-1,15-dioxo-1,2-dihydro-2,7-(epoxypentadeca[1,11,13]trienoimino)furo[2″,3″:7′,8′]naphtho[1′,2′:4,5]imidazo[1,2-a]pyrid; or, 2S-Acetyloxy-5,6,21,23-tetrahydroxy-27-methoxy-2,4,11, 16,20,22,24,26-octamethyl-2,7-(epoxypentoeleca(1,11,13)trienimino)benzofuro[4,5-e]pyride[1,2-a]benzimidazole-1,15(2H)-dione; 80621-81-4; or, enantiomers or stereoisomers thereof) (optionally a XIFAXAN™ XIFAXANTA™, RITACOL™, FATROXIMIN™, XIFAXSAN™, RIFAXIMINUM™, RIFAXIMINUN™, RIFAXIMINE™, RIFAXIMIN™, RIFAXIDIN™, RIFAXIMINA™, RIFAMYCIN™, VETRANAL™, or NORMIX™), a polymorphic form of a rifaximin or a rifaximin equivalent thereof, an extended intestinal release (EIR) rifaximin, a rifamycin derivative, a rifampicin (or rifampin) (optionally RIFADIN™), a rifabutin (optionally MYCOBUTIN™), a rifapentin (optionally PRIFTIN™), a rifalazil, a bicozamycin, a pyrido-imidazo rifamycin, a dehydro-rifaximin, a rifaximin histidine, a rifaximin tryptophan, an 11-desmethyl-rifaximin (e.g., an 11-desmethyl NORMIX™), a rifaximin beta-cyclodextrin, or equivalents thereof or a mixture or a combination thereof,
(ii) vancomycin, neomycin, tobramycin, paromomycin or streptomycin,
(iii) gentamicin, streptomycin, ridinilazole, teicoplanin, streptomycin and neomycin, fidaxomicin, ramoplanin, surotomycin, capozide, a partially absorbed agent, optionally comprising a tinidazole, metronidazole, nitazoxanide, amoxicillin, tetracyclines, ornidazole, secnidazole, ciprofloxacin or an ansamycin, or
(iv) an antibiotic or drug as listed in Table 1; or
(b) an antibiotic or drug of (a), and at least one additional antimicrobial or antibiotic agent,
wherein optionally for step (a) or (b) the rifaximin or rifaximin polymorphic form thereof or rifaximin equivalent comprises:
(i) a rifaximin, a rifaximin polymorph or a rifaximin equivalent as described in U.S. Pat. No. 9,273,066, optionally comprising a polymorphic form zeta of rifaximin exhibiting an X-ray powder diffraction pattern having characteristic peaks expressed in degrees 2 theta (+/−0.20 degree theta) comprising 4.69, 7.63, 12.52, 13.87;
(ii) a 25-desacetyl rifaximin, or a rifaximin, a rifaximin polymorph or a rifaximin equivalent, as described in U.S. Pat. No. 9,364,467, optionally comprising a 25-desacetyl rifaximin or a pharmaceutically acceptable salt thereof (optionally a sodium, potassium, calcium, magnesium, ammonium, or chlorine pharmaceutically acceptable salt of 25-desacetyl rifaximin), wherein optionally the 25-desacetyl rifaximin has the formula:

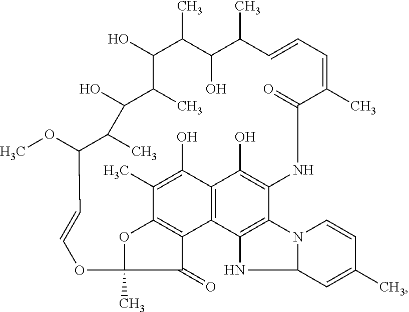 [SMILES: COC1/C=C/O[C@@]2(C)Oc3c(C)c(O)c4c(O)c(c5c(c4c3C2=O)NC2C=C(C)C=CN52)NC(=O)/C(C)=C\C=C\C(C)C(O)C(C)C(O)C(C)C(O)C1C]

(iii) a rifaximin, a rifaximin polymorph or a rifaximin equivalent as described in U.S. Pat. No. 9,546,183, optionally comprising a polymorphic Form B of rifaximin exhibiting an X-ray powder diffraction pattern having characteristic peaks expressed in degrees 2 theta (+/−0.20 degree theta) at 5.24, 6.84, 7.74, 8.71, 10.16, and 12.21,
(iv) a rifaximin amorphous form or a rifaximin equivalent as described in U.S. Pat. No. 9,700,545, optionally comprising an amorphous form of rifaximin exhibiting an X-ray powder diffraction pattern having characteristic peaks expressed in degrees: (1) 2 theta (+/−0.20 degree theta) at 7.3, 11.3-17.8, and 15.8 degrees 2 theta; 2 theta (+/−0.20 degree theta) at 5.1-10.1, 11.3-17.8, and 15.8 degrees 2 theta; or (3) 2 theta (+/−0.20 degree theta) at 5.1-10.1, 7.3, and 11.3-17.8 degrees 2 theta,
(v) a rifaximin, a rifaximin polymorph or a rifaximin equivalent as described in U.S. Pat. No. 9,359,374, or U.S. Pat. No. 9,725,466, optionally comprising a polymorphic form APO-III of rifaximin characterized by a PXRD diffractogram comprising peaks, in terms of degrees 2-theta, at approximately 7.1, 8.4, 11.6, 13.1, 18.5, 18.8, and 25.0,
(vi) a rifaximin, a rifaximin polymorph or a rifaximin equivalent as described in U.S. Pat. No. 9,421,195,
(vii) a rifaximin, a rifaximin polymorph or a rifaximin equivalent as described in U.S. Pat. No. 7,045,620, optionally a crystalline polymorphous form of a rifaximin, a rifaximin polymorph or a rifaximin equivalent; and/or
(viii) a controlled-release or spray-dried rifaximin, rifaximin polymorph or rifaximin equivalent as described in U.S. Pat. No. 9,498,442, optionally rifaximin, rifaximin polymorph or rifaximin equivalent characterized by an X-Ray diffraction spectrum showing diffraction halo peaks in the range 7.75 degree+−0.0.2-18.33 degree±0.0.2, 2 theta, with maximum at about 7.75 degree±0.0.2 and in the range 14.54 degree±0.0.2 and 18.33 degree±0.0.2, 2 theta.
2. The method of form 1, wherein the IBD further comprises or is associated with a condition or side effect comprising diarrhoea, rectal bleeding, mucus, urgency, incontinence, nocturnal diarrhoea; together with lower abdominal pain, weight loss, excessive gas production, bloating, loss of appetite, joint pains/symptoms, and optionally administration of the formulation, pharmaceutical preparation, therapeutic combination or pharmaceutical composition treats, ameliorates, reverses, causes the remission of, and/or prevents (acts as a prophylaxis) one, several or all of these conditions or side effects.
3. The method of form 1 or form 2, wherein the formulation, pharmaceutical preparation, therapeutic combination or pharmaceutical composition further comprises at least one additional antimicrobial or antibiotic agent, or further comprises a drug or a probiotic.
4. The method of form 3, wherein the at least one additional antimicrobial or antibiotic agent comprises a vancomycin, a metronidazole (optionally FLAGYL™, METRO™), a tinidazole (optionally FASIGYN™, SIMPLOTAN™, TINDAMAX™), an ornidazole (optionally XYNOR™), a secnidazole (optionally FLAGENTYL™, SINDOSE™, SECNIL™) an antibiotic or drug as listed in Table 1, or a combination thereof.
5. The method of any of the preceding forms, wherein the at least one additional antimicrobial or antibiotic agent comprises: an antibiotic or antibacterial agent from one or more of the following classes selected from: tetracyclines, penicillins, macrolides, quinolones, chloramphenicol, rifamycins, sulphonamides, co-trimoxazole, and oxazolidinones.
6. The method of any of the preceding forms, wherein the at least one additional antimicrobial or antibiotic agent comprises: a doxycycline, chlortetracycline, tetracycline hydrochloride, oxytetracycline, demeclocycline, methacycline, minocycline, penicillin, amoxycillin, erythromycin, clarithromycin, roxithromycin, azithromycin, spiramycin, oleandomycin, josamycin, kitsamysin, flurithromycin, nalidixic acid, oxolinic acid, norfloxacin, perfloxacin, amifloxacin, ofloxacin, moxifloxacin, ciprofloxacin, sparfloxacin, levofloxacin, rifabutin, rifampicin, rifapentin, rifalazil, sulfisoxazole, sulfamethoxazole, sulfadiazine, sulfadoxine, sulfasalazine, sulfaphenazole, dapsone, sulfacytidine, linezolid or any combination thereof.
7. The method of any of the preceding forms, wherein the at least one additional antimicrobial or antibiotic agent comprises:
an ampicillin, a sulbactama tetracycline, a cephalosporin, a carbapenem, an imipenem, a meropenem, a monobactam, a lincosamide, a clindamycin, a quinolone, a fluoroquinolone, a sulphonamide, a fradicin, a nitroimidazole, a metronidazole, a tinidazole, a secnidazole, an anti-Clostridial agent, or a ramoplanan,
an aminoglycoside antibiotic, a gentamycin, a neomycin, a streptomycin, a paromomycin, a verdamicin, a mutamicin, a sisomicin, a netilmicin, a retymicin, a kanamycin, an amphenicol, an ansamycin, a beta-lactam (β-lactam) antibiotic, a carbapenem, a cephalosporin, a cephamycin, a monobactam, an oxacephem, a lincosamide antibiotic, a clindamycin, or a lincomycin,
a glycopeptide antibiotic, a vancomycin, a teicoplanin, a telavancin, a bleomycin, a ramoplanin, a decaplanin, a polypeptide antibiotic, an actinomycin, an actinomycin D, a bacitracin, a bacitracin, a tetracycline, a 2,4-diaminopyrimidine class antibiotic, a clavacin, a clairformin, a claviform, an expansine, a clavatin, an expansin, a gigantin, a leucopin, a patuline or a patulin), or
an equivalent thereof or a combination thereof.
8. The method of any of the preceding forms, wherein the at least one additional antimicrobial or antibiotic agent comprises:
(i) a rifaximin (optionally a XIFAXAN™, XIFAXANTA™ or NORMIX™), RITACOL™), FATROXIMIN™), XIFAXSAN™), RIFAXIMINUM™), RIFAXIMINUN™), RIFAXIMINE™), RIFAXIMIN™), RIFAXIDIN™), RIFAXIMINA™, RIFAMYCIN™, a polymorphic form of a rifaximin or a rifaximin equivalent thereof, an extended intestinal release (EIR) rifaximin, a rifamycin derivative, a rifampicin (or rifampin) (optionally RIFADIN™), a rifabutin (optionally MYCOBUTIN™), a rifapentin (optionally PRIFTIN™), a rifalazil, a bicozamycin, a pyrido-imidazo rifamycin, a dehydro-rifaximin, a rifaximin histidine, a rifaximin tryptophan, an 11-desmethyl-rifaximin (e.g., an 11-desmethyl NORMIX™), a rifaximin beta-cyclodextrin, or equivalents thereof or a mixture or a combination thereof,
(ii) vancomycin, neomycin, tobramycin, paromomycin or streptomycin,
(iii) gentamicin, streptomycin, ridinilazole, teicoplanin, streptomycin and neomycin, fidaxomicin, ramoplanin, surotomycin, capozide, a partially absorbed agent, optionally comprising a tinidazole, metronidazole, nitazoxanide, amoxicillin, tetracyclines, ornidazole, secnidazole, ciprofloxacin or an ansamycin, or
(iv) an antibiotic or drug as listed in Table 1, or
(v) any combination thereof.
9. The method of any of the preceding forms, wherein the antimicrobial or antibiotic agent comprises a three-drug therapeutic combination comprising:
rifaximin, tinidazole and nitazoxanide; rifaximin, tinidazole and oral tobramycin; rifaximin, amoxicillin and metronidazole; rifaximin, paromomycin and nitazoxanide; rifaximin, paromomycin and vancomycin; rifaximin, tinidazole and paromomycin; rifaximin, ridinilazole and tobramycin; rifaximin, ridinilazole and paromomycin; or rifaximin, ridinilazole and nitazoxanide; and optionally teicoplanin is substituted for any one of the second or third drug in this 3-drug combination list.
10. The method of any one of forms 1 to 8, wherein the formulation, pharmaceutical preparation, therapeutic combination, or pharmaceutical composition comprises a rifamycin, a nitroimidazole, and a tetracycline antibiotic.
11. The method of form 10 wherein the rifamycin is rifampicin, the nitroimidazole is secnidazole, and the tetracycline antibiotic is doxycycline.
12. The method of any one of forms 1 to 8, wherein the formulation, pharmaceutical preparation, therapeutic combination, or pharmaceutical composition comprises a rifamycin, a nitroimidazole, and a thiazolide.
13. The method of form 12, wherein the rifamycin is rifaximin, the nitroimidazole is tinidazole, and the thiazolide is nitazoxanide.
14. The method of any one of forms 1 to 8, wherein the formulation, pharmaceutical preparation, therapeutic combination, or pharmaceutical composition comprises fosfomycin, a nitroimidazole, and a tetracycline antibiotic.
15. The method of form 14, wherein the nitroimidazole is metronidazole, and the tetracycline antibiotic is doxycycline.
16. The method of any of the preceding forms, wherein the individual exhibits at least an about 5% to 10%, or 10% to 20%, or 20% to 30%, or 30% to 40%, or 40% to 50%, or 50% to 60%, or 70% to 80%, or 80% to 90%, or substantially complete, reduction in IBD symptoms or side effects or severity after administration of the formulation, pharmaceutical preparation, therapeutic combination or pharmaceutical composition to the individual in need thereof as compared to before initiating the administration.
17. The method of form 16, wherein the at least an about 5% to 10%, or 10% to 20%, or 20% to 30%, or 30% to 40%, or 40% to 50%, or 50% to 60%, or 70% to 80%, or 80% to 90%, or substantially complete, reduction in IBD symptoms or side effects or severity is achieved after about 1, 2 or 3 or more weeks, or after about 1 to 2 months, or after about 2 to 6 months, of initiating the administration.
18. The method of form 16, wherein the at least an about 5% to 10%, or 10% to 20%, or 20% to 30%, or 30% to 40%, or 40% to 50%, or 50% to 60%, or 70% to 80%, or 80% to 90%, or substantially complete, reduction in IBD symptoms or side effects or severity is maintained for at least about 4 to 8 weeks, or 2 to 6 months, after discontinuing the administration to the individual.
19. The method of any of the preceding forms, wherein the formulation, pharmaceutical preparation, therapeutic combination or pharmaceutical composition is formulated as a chewable delivery vehicle, a gum, a gummy, a candy, a lozenge, an ice cream or an ice, a yogurt or a drink.
20. The method of any of the preceding forms, wherein a unit dosage of the formulation, pharmaceutical preparation, therapeutic combination or pharmaceutical composition is a pediatric unit dosage, and optionally the unit dosage is between about 10 mg and 1100 mgm, or between about between about 40 mg and 4,000 mgm, or is about 10, 20, 30, 40, 50, 60, 70, 75, 80, 90, 100, 125, 150, 175, 200, 225, 250, 275, 300, 325, 350, 375, 400, 425, 450, 475, 500, 600, 700, 750, 800, 900, 1000, 1100, 1500, 2000, 2500, 3000, 3500, 4000, or more mg per unit dose, which optionally can be administered once a day, bid or tid, or a four times a day, five times a day or six times a day or more, regimen.
21. The method of any of the preceding forms, wherein a daily dosage of the formulation, pharmaceutical preparation, therapeutic combination or pharmaceutical composition is about 50, 75, 100, 125, 150, 175, 200, 225, 250, 275, 300, 325, 350, 375, 400, 425, 450, 475, 500, 600, 700, 750, 800, 900, 1000, 1100, 1500, 2000, 2500, 3000, 3500, 4000, or more mg per day, or between about 100 and 1100 mgm per day total, or between about 400 and 4000 mg per day, which optionally can be administered in a once a day, bid or tid, or four times a day, five times a day or six times a day or more, regimen.
22. The method of any of the preceding forms, wherein a unit dosage of the formulation, pharmaceutical preparation, therapeutic combination or pharmaceutical composition is set for (the daily dosage is set for) bid (twice a day), tid (three times a day), four times a day, five times a day or six times a day or more, with the unit dosage and daily dosage adjusted to be: about 1000 mg/70 kg a day, or about 14 mg/kg a day, for an adult median dose per day; or for a pediatric dosage about 350 mg/25 kg a day, or about 15 to 16 mg/kg, a day; or equivalent.
23. The method of any of the preceding forms, wherein the daily dosage of the formulation, pharmaceutical preparation, therapeutic combination or pharmaceutical composition is about 25 mg to 20 grams (gm) bid, or about 400, 425, 450, 475, 500, 600, 700, 750, 800, 900, 1000, 1100, 1200, 1500, 2000, 2500, 3000, 3500, 4000, or more mgm once a day, bid or tid, or four times a day, five times a day or six times a day or more.
24. The method of any of the preceding forms, wherein the daily dosage of the formulation, pharmaceutical preparation, therapeutic combination or pharmaceutical composition, or one ingredient of the formulation, pharmaceutical preparation, therapeutic combination or pharmaceutical composition, is increased or “ramped up” every week, or every other week, by about 25, 50, 75, 100, 125, 150, 175, 200, 225, 250, 275, 300, 325, 350, 375, 400, 425, 450, 475, 1500, 2000, 2500, 3000, 3500, 4000, or more or more mg per week, or every other week,
and optionally this “ramping up” or increasing of dosages continues for about a month, about 6 months or about a year, or until symptoms of IBD significantly diminish or abate, or significantly diminish or abate without need for administration of the formulation, the pharmaceutical or the pharmaceutical preparation.
25. The method of any of the preceding forms, wherein the formulation, the pharmaceutical or the pharmaceutical preparation further comprises a flavoring or a sweetening agent, an aspartamine, a stevia, monk fruit, a sucralose, a saccharin, a cyclamate, a xylitol, a vanilla, an artificial vanilla or chocolate or strawberry flavor, an artificial chocolate essence, or a mixture or combination thereof.
26. The method of any of the preceding forms, wherein the formulation, pharmaceutical preparation, therapeutic combination or pharmaceutical composition further comprises a preservative, a benzoic acid or a potassium sorbate.
27. The method of any of the preceding forms, wherein the formulation, pharmaceutical preparation, therapeutic combination or pharmaceutical composition further comprises, or has added to: at least one probiotic or prebiotic,
wherein optionally the prebiotic comprises an inulin, lactulose, extracts of artichoke, chicory root, oats, barley, various legumes, garlic, kale, beans or flacks or an herb,
and optionally the probiotic comprises a cultured or stool-extracted microorganism or bacteria, or a bacterial component, and optionally the probiotic bacteria or bacterial component comprises or is derived from a non-pathogenic Clostridia, a Bacteroidetes, a Firmicutes, a Lactobacilli, a Bifidobacteria, an E. coli, a Strep fecalis, an Actinobacteria, a Proteobacteria, a Verruco-microbia, a Fusobacteria, a Cyanobacteria, a Spirochetes and a Lentisphaerae, and equivalents.
28. The method of any of the preceding forms, wherein the formulation, pharmaceutical preparation, therapeutic combination or pharmaceutical composition further comprises, or has added to: at least one congealing agent, wherein optionally the congealing agent comprises an arrowroot or a plant starch, a powdered flour, a powdered potato or potato starch, an absorbant polymer, an Absorbable Modified Polymer, and/or a corn flour or a corn starch.
29. The method of any of the preceding forms, wherein the formulation, pharmaceutical preparation, therapeutic combination or pharmaceutical composition further comprises an additive selected from one or more of a saline, a media, a defoaming agent, a surfactant agent, a lubricant, an acid neutralizer, a marker, a cell marker, a drug, an antibiotic, a contrast agent, a dispersal agent, a buffer or a buffering agent, a sweetening agent, a debittering agent, a flavoring agent, a pH stabilizer, an acidifying agent, a preservative, a desweetening agent and/or coloring agent, vitamin, mineral and/or dietary supplement, or a prebiotic nutrient.
30. The method of any of the preceding forms, wherein the formulation, pharmaceutical preparation, therapeutic combination or pharmaceutical composition further comprises, or has added to: at least one Biofilm Disrupting Compound, wherein optionally the biofilm disrupting compound comprises an enzyme, a deoxyribonuclease (DNase), N-acetylcysteine, an auranofin, an alginate lyase, glycoside hydrolase dispersin B; a Quorum-sensing inhibitor, a ribonucleic acid III inhibiting peptide, Salvadora persica extracts, Competence-stimulating peptide, Patulin and penicillic acid; peptides—cathelicidin-derived peptides, small lytic peptide, PTP-7, Nitric oxide, neo-emulsions; ozone, lytic bacteriophages, lactoferrin, xylitol hydrogel, synthetic iron chelators, cranberry components, curcumin, silver nanoparticles, Acetyl-11-keto-β-boswellic acid (AKBA), barley coffee components, probiotics, sinefungin, S-adenosylmethionine, S-adenosyl-homocysteine, Delisea furanones, N-sulfonyl homoserine lactones or any combination thereof.
31. The method of any of the preceding forms, wherein the formulation, pharmaceutical preparation, therapeutic combination or pharmaceutical composition is formulated as a delayed or gradual enteric release composition or formulation, and optionally the formulation comprises a gastro-resistant coating designed to dissolve at a pH of 7 in the terminal ileum, e.g., an active ingredient is coated with an acrylic based resin or equivalent, e.g., a poly(meth)acrylate, e.g. a methacrylic acid copolymer B, NF, which dissolves at pH 7 or greater, e.g., comprises a multimatrix (MMX) formulation.
32. The method of any of the preceding forms, wherein the formulation, pharmaceutical preparation, therapeutic combination or pharmaceutical composition is contained in a delivery vehicle, product of manufacture, container, syringe, device or bag.
33. The method of any of the preceding forms, wherein the formulation, pharmaceutical preparation, therapeutic combination or pharmaceutical composition is initially manufactured or formulated as a liquid, a suspension, a gel, a geltab, a semisolid, a tablet, a sachet, a lozenge or a capsule, or as an enteral formulation, or re-formulated for final delivery as a liquid, a suspension, a gel, a geltab, a semisolid, a tablet, a sachet, a lozenge or a capsule, or as an enteral formulation.

In a second aspect, forms of the invention, may include the following.

1. Use of a pharmaceutical composition comprising or consisting of:
(a) (i) a rifaximin (or, In-25-yl acetate; (16Z,18E,28E)-5,6,21,23-tetrahydroxy-27-methoxy-2,4,11,16,20,22,24,26-octamethyl-1,15-dioxo-1,2-dihydro-2,7-(epoxypentadeca[1,11,13]trienoimino)furo[2″,3″:7′,8′]naphtho[1′,2′:4,5]imidazo[1,2-a]pyrid; or, 2S-Acetyloxy-5,6,21,23-tetrahydroxy-27-methoxy-2,4,11, 16,20,22,24,26-octamethyl-2,7-(epoxypentoeleca(1,11,13)trienimino)benzofuro[4,5-e]pyride[1,2-a]benzimidazole-1,15(2H)-dione; 80621-81-4; or, enantiomers or stereoisomers thereof) (optionally a XIFAXAN™, XIFAXANTA™, RITACOL™, FATROXIMIN™, XIFAXSAN™, RIFAXIMINUM™, RIFAXIMINUN™, RIFAXIMINE™, RIFAXIMIN™, RIFAXIDIN™, RIFAXIMINA™, RIFAMYCIN™, VETRANAL™, or NORMIX™), a polymorphic form of a rifaximin or a rifaximin equivalent thereof, an extended intestinal release (EIR) rifaximin, a rifamycin derivative, a rifampicin (or rifampin) (optionally RIFADIN™), a rifabutin (optionally MYCOBUTIN™), a rifapentin (optionally PRIFTIN™), a rifalazil, a bicozamycin, a pyrido-imidazo rifamycin, a dehydro-rifaximin, a rifaximin histidine, a rifaximin tryptophan, an 11-desmethyl-rifaximin (e.g., an 11-desmethyl NORMIX™), a rifaximin beta-cyclodextrin, fosfomycin, or equivalents thereof or a mixture or a combination thereof,
(ii) vancomycin, neomycin, tobramycin, paromomycin or streptomycin,
(iii) gentamicin, streptomycin, ridinilazole, teicoplanin, streptomycin and neomycin, fidaxomicin, ramoplanin, surotomycin, capozide, a partially absorbed agent, optionally comprising a tinidazole, metronidazole, nitazoxanide, amoxicillin, tetracyclines, ornidazole, secnidazole, ciprofloxacin or an ansamycin, or
(iv) an antibiotic or drug as listed in Table 1; or
(b) an antibiotic or drug of (a), and at least one additional antimicrobial or antibiotic agent,
wherein optionally for (a) or (b) the rifaximin or rifaximin polymorphic form thereof or rifaximin equivalent comprises:
(i) a rifaximin, a rifaximin polymorph or a rifaximin equivalent as described in U.S. Pat. No. 9,273,066, optionally comprising a polymorphic form zeta of rifaximin exhibiting an X-ray powder diffraction pattern having characteristic peaks expressed in degrees 2 theta (+/−0.20 degree theta) comprising 4.69, 7.63, 12.52, 13.87;
(ii) a 25-desacetyl rifaximin, or a rifaximin, a rifaximin polymorph or a rifaximin equivalent, as described in U.S. Pat. No. 9,364,467, optionally comprising a 25-desacetyl rifaximin or a pharmaceutically acceptable salt thereof (optionally a sodium, potassium, calcium, magnesium, ammonium, or chlorine pharmaceutically acceptable salt of 25-desacetyl rifaximin), wherein optionally the 25-desacetyl rifaximin has the formula:

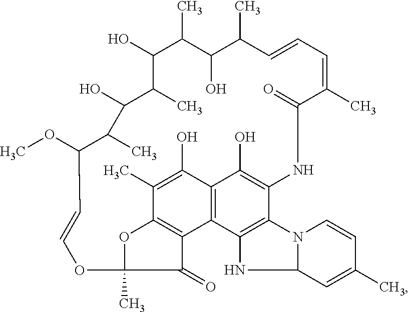 [SMILES: COC1/C=C/O[C@@]2(C)Oc3c(C)c(O)c4c(O)c(c5c(c4c3C2=O)NC2C=C(C)C=CN52)NC(=O)/C(C)=C\C=C\C(C)C(O)C(C)C(O)C(C)C(O)C1C]

(iii) a rifaximin, a rifaximin polymorph or a rifaximin equivalent as described in U.S. Pat. No. 9,546,183, optionally comprising a polymorphic Form B of rifaximin exhibiting an X-ray powder diffraction pattern having characteristic peaks expressed in degrees 2 theta (+/−0.20 degree theta) at 5.24, 6.84, 7.74, 8.71, 10.16, and 12.21,
(iv) a rifaximin amorphous form or a rifaximin equivalent as described in U.S. Pat. No. 9,700,545, optionally comprising an amorphous form of rifaximin exhibiting an X-ray powder diffraction pattern having characteristic peaks expressed in degrees: (1) 2 theta (+/−0.20 degree theta) at 7.3, 11.3-17.8, and 15.8 degrees 2 theta; 2 theta (+/−0.20 degree theta) at 5.1-10.1, 11.3-17.8, and 15.8 degrees 2 theta; or (3) 2 theta (+/−0.20 degree theta) at 5.1-10.1, 7.3, and 11.3-17.8 degrees 2 theta,
(v) a rifaximin, a rifaximin polymorph or a rifaximin equivalent as described in U.S. Pat. No. 9,359,374, or U.S. Pat. No. 9,725,466, optionally comprising a polymorphic form APO-III of rifaximin characterized by a PXRD diffractogram comprising peaks, in terms of degrees 2-theta, at approximately 7.1, 8.4, 11.6, 13.1, 18.5, 18.8, and 25.0,
(vi) a rifaximin, a rifaximin polymorph or a rifaximin equivalent as described in U.S. Pat. No. 9,421,195,
(vii) a rifaximin, a rifaximin polymorph or a rifaximin equivalent as described in U.S. Pat. No. 7,045,620, optionally a crystalline polymorphous form of a rifaximin, a rifaximin polymorph or a rifaximin equivalent; and/or
(viii) a controlled-release or spray-dried rifaximin, rifaximin polymorph or rifaximin equivalent as described in U.S. Pat. No. 9,498,442, optionally rifaximin, rifaximin polymorph or rifaximin equivalent characterized by an X-Ray diffraction spectrum showing diffraction halo peaks in the range 7.75 degree+−0.0.2-18.33 degree±0.0.2, 2 theta, with maximum at about 7.75 degree±0.0.2 and in the range 14.54 degree±0.0.2 and 18.33 degree±0.0.2, 2 theta,
in the manufacture of a medicament for treating, ameliorating, reversing, causing the remission of, and/or preventing (acting as a prophylaxis, or preventing the initiation of) an inflammatory bowel disease or disorder (IBD); ulcerative colitis; Crohn's disease; J-pouch; fistulising Crohn's disease; a colitis which can be microscopic, lymphocytic or collagenous; an eosinophilic colitis; indeterminate colitis; idiopathic colitis; diverticulosis; diverticulitis; relapsing diverticulitis; constipation associated inflammatory bowel disease and/or small intestinal bacterial overgrowth; irritable bowel syndrome (IBS) with or without diarrhoea, constipation or pain predominant IBS; periodontitis; rheumatoid arthritis; respiratory infections, appendicitis, vascular disorders such as thrombophlebitis; bacteremia; osteomyelitis; septic shock; Alzheimer's disease; Lemierre syndrome (postanginal sepsis); colonic polyps; or adenomas (optionally hyperplastic, adenomatous or serrated adenomas); or
preventing the growth of, or slowing the progression or recurrence of, colonic polyps or adenomas, bowl cancer, or metastases optionally preventing the initiation of or recurrence of or promotion of bowl cancer or metastasis; pharyngitis; otitis; or sinusitis; or
treating, ameliorating, reversing, causing the remission of, and/or preventing any disease, symptom or condition caused or exacerbated by a Fusobacteria infection, e.g., a F. nucleatum, F. varium, F. simae, F. periodonticum, F. equimun, or F. Necrogenes) infection,
in an individual in need thereof.
2. The use of form 1, wherein the IBD further comprises or is associated with a condition or side effect comprising diarrhoea, rectal bleeding, mucus, urgency, incontinence, nocturnal diarrhoea; together with lower abdominal pain, weight loss, excessive gas production, bloating, loss of appetite, joint pains/symptoms, and optionally administration of the formulation, pharmaceutical preparation, therapeutic combination or pharmaceutical composition treats, ameliorates, reverses, causes the remission of, and/or prevents (acts as a prophylaxis) one, several or all of these conditions or side effects.
3. The use of form 1 or form 2, wherein the pharmaceutical composition further comprises at least one additional antimicrobial or antibiotic agent, or further comprises a drug or a probiotic.
4. The use of form 3, wherein the at least one additional antimicrobial or antibiotic agent comprises a vancomycin, a metronidazole (optionally FLAGYL™, METRO™), a tinidazole (optionally FASIGYN™, SIMPLOTAN™, TINDAMAX™), an ornidazole (optionally XYNOR™), a secnidazole (optionally FLAGENTYL™, SINDOSE™, SECNIL™), an antibiotic or drug as listed in Table 1, or a combination thereof.
5. The use of any of the preceding forms, wherein the at least one additional antimicrobial or antibiotic agent comprises: an antibiotic or antibacterial agent from one or more of the following classes selected from: tetracyclines, penicillins, macrolides, quinolones, chloramphenicol, rifamycins, sulphonamides, co-trimoxazole, and oxazolidinones.
6. The use of any of the preceding forms, wherein the at least one additional antimicrobial or antibiotic agent comprises: a doxycycline, chlortetracycline, tetracycline hydrochloride, oxytetracycline, demeclocycline, methacycline, minocycline, penicillin, amoxycillin, erythromycin, clarithromycin, roxithromycin, azithromycin, spiramycin, oleandomycin, josamycin, kitsamysin, flurithromycin, nalidixic acid, oxolinic acid, norfloxacin, perfloxacin, amifloxacin, ofloxacin, moxifloxacin, ciprofloxacin, sparfloxacin, levofloxacin, rifabutin, rifampicin, rifapentin, rifalazil, sulfisoxazole, sulfamethoxazole, sulfadiazine, sulfadoxine, sulfasalazine, sulfaphenazole, dapsone, sulfacytidine, linezolid or any combination thereof.
7. The use of any of the preceding forms, wherein the at least one additional antimicrobial or antibiotic agent comprises:
an ampicillin, a sulbactama tetracycline, a cephalosporin, a carbapenem, an imipenem, a meropenem, a monobactam, a lincosamide, a clindamycin, a quinolone, a fluoroquinolone, a sulphonamide, a fradicin, a nitroimidazole, a metronidazole, a tinidazole, a secnidazole, an anti-Clostridial agent, or a ramoplanan,
an aminoglycoside antibiotic, a gentamycin, a neomycin, a streptomycin, a paromomycin, a verdamicin, a mutamicin, a sisomicin, a netilmicin, a retymicin, a kanamycin, an amphenicol, an ansamycin, a beta-lactam (β-lactam) antibiotic, a carbapenem, a cephalosporin, a cephamycin, a monobactam, an oxacephem, a lincosamide antibiotic, a clindamycin, or a lincomycin, a glycopeptide antibiotic, a vancomycin, a teicoplanin, a telavancin, a bleomycin, a ramoplanin, a decaplanin, a polypeptide antibiotic, an actinomycin, an actinomycin D, a bacitracin, a bacitracin, a tetracycline, a 2,4-diaminopyrimidine class antibiotic, a clavacin, a clairformin, a claviform, an expansine, a clavatin, an expansin, a gigantin, a leucopin, a patuline or a patulin), or
an equivalent thereof or a combination thereof.
8. The use of any of the preceding forms, wherein the at least one additional antimicrobial or antibiotic agent comprises:
(i) a rifaximin (optionally a XIFAXAN™, XIFAXANTA™ or NORMIX™), RITACOL™), FATROXIMIN™), XIFAXSAN™), RIFAXIMINUM™), RIFAXIMINUN™), RIFAXIMINE™), RIFAXIMIN™), RIFAXIDIN™), RIFAXIMINA™, RIFAMYCIN™, a polymorphic form of a rifaximin or a rifaximin equivalent thereof, an extended intestinal release (EIR) rifaximin, a rifamycin derivative, a rifampicin (or rifampin) (optionally RIFADIN™), a rifabutin (optionally MYCOBUTIN™), a rifapentin (optionally PRIFTIN™), a rifalazil, a bicozamycin, a pyrido-imidazo rifamycin, a dehydro-rifaximin, a rifaximin histidine, a rifaximin tryptophan, an 11-desmethyl-rifaximin (e.g., an 11-desmethyl NORMIX™), a rifaximin beta-cyclodextrin, or equivalents thereof or a mixture or a combination thereof,
(ii) vancomycin, neomycin, tobramycin, paromomycin or streptomycin,
(iii) gentamicin, streptomycin, ridinilazole, teicoplanin, streptomycin and neomycin, fidaxomicin, ramoplanin, surotomycin, capozide, a partially absorbed agent, optionally comprising a tinidazole, metronidazole, nitazoxanide, amoxicillin, tetracyclines, ornidazole, secnidazole, ciprofloxacin or an ansamycin, or
(iv) an antibiotic or drug as listed in Table 1, or
(v) any combination thereof.
9. The use of any of the preceding forms, wherein the antimicrobial or antibiotic agent comprises a three-drug therapeutic combination comprising:
rifaximin, tinidazole and nitazoxanide; rifaximin, tinidazole and oral tobramycin; rifaximin, amoxicillin and metronidazole; rifaximin, paromomycin and nitazoxanide; rifaximin, paromomycin and vancomycin; rifaximin, tinidazole and paromomycin; rifaximin, ridinilazole and tobramycin; rifaximin, ridinilazole and paromomycin; or rifaximin, ridinilazole and nitazoxanide; and optionally teicoplanin is substituted for any one of the second or third drug in this 3-drug combination list.
10. The use of any one of forms 1 to 8, wherein the pharmaceutical composition comprises a rifamycin, a nitroimidazole, and a tetracycline antibiotic.
11. The use of form 10 wherein the rifamycin is rifampicin, the nitroimidazole is secnidazole, and the tetracycline antibiotic is doxycycline.
12. The use of any one of forms 1 to 8, wherein the pharmaceutical composition comprises a rifamycin, a nitroimidazole, and a thiazolide.
13. The use of form 12, wherein the rifamycin is rifaximin, the nitroimidazole is tinidazole, and the thiazolide is nitazoxanide.
14. The use of any one of forms 1 to 8, wherein the pharmaceutical composition comprises fosfomycin, a nitroimidazole, and a tetracycline antibiotic.
15. The use of form 14, wherein the nitroimidazole is metronidazole, and the tetracycline antibiotic is doxycycline.
16. The use of any of the preceding forms, wherein the individual exhibits at least an about 5% to 10%, or 10% to 20%, or 20% to 30%, or 30% to 40%, or 40% to 50%, or 50% to 60%, or 70% to 80%, or 80% to 90%, or substantially complete, reduction in IBD symptoms or side effects or severity after administration of the medicament, as compared to before initiating the administration.
17. The use of form 16, wherein the at least an about 5% to 10%, or 10% to 20%, or 20% to 30%, or 30% to 40%, or 40% to 50%, or 50% to 60%, or 70% to 80%, or 80% to 90%, or substantially complete, reduction in IBD symptoms or side effects or severity is achieved after about 1, 2 or 3 or more weeks, or after about 1 to 2 months, or after about 2 to 6 months, of initiating the administration.
18. The use of form 16, wherein the at least an about 5% to 10%, or 10% to 20%, or 20% to 30%, or 30% to 40%, or 40% to 50%, or 50% to 60%, or 70% to 80%, or 80% to 90%, or substantially complete, reduction in IBD symptoms or side effects or severity is maintained for at least about 4 to 8 weeks, or 2 to 6 months, after discontinuing the administration to the individual.
19. The use of any of the preceding forms, wherein the medicament is formulated as a chewable delivery vehicle, a gum, a gummy, a candy, a lozenge, an ice cream or an ice, a yogurt or a drink.
20. The use of any of the preceding forms, wherein a unit dosage of the medicament is a pediatric unit dosage, and optionally the unit dosage is between about 10 mg and 1100 mgm, or between about between about 40 mg and 4,000 mgm, or is about 10, 20, 30, 40, 50, 60, 70, 75, 80, 90, 100, 125, 150, 175, 200, 225, 250, 275, 300, 325, 350, 375, 400, 425, 450, 475, 500, 600, 700, 750, 800, 900, 1000, 1100, 1500, 2000, 2500, 3000, 3500, 4000, or more mg per unit dose, which optionally can be administered once a day, bid or tid, or a four times a day, five times a day or six times a day or more, regimen.
21. The use of any of the preceding forms, wherein a daily dosage of the medicament is about 50, 75, 100, 125, 150, 175, 200, 225, 250, 275, 300, 325, 350, 375, 400, 425, 450, 475, 500, 600, 700, 750, 800, 900, 1000, 1100, 1500, 2000, 2500, 3000, 3500, 4000, or more mg per day, or between about 100 and 1100 mgm per day total, or between about 400 and 4000 mg per day, which optionally can be administered in a once a day, bid or tid, or four times a day, five times a day or six times a day or more, regimen.
22. The use of any of the preceding forms, wherein a unit dosage of the medicament is set for (the daily dosage is set for) bid (twice a day), tid (three times a day), four times a day, five times a day or six times a day or more, with the unit dosage and daily dosage adjusted to be: about 1000 mg/70 kg a day, or about 14 mg/kg a day, for an adult median dose per day; or for a pediatric dosage about 350 mg/25 kg a day, or about 15 to 16 mg/kg, a day; or equivalent.
23. The use of any of the preceding forms, wherein the daily dosage of the medicament is about 25 mg to 20 grams (gm) bid, or about 400, 425, 450, 475, 500, 600, 700, 750, 800, 900, 1000, 1100, 1200, 1500, 2000, 2500, 3000, 3500, 4000, or more mgm once a day, bid or tid, or four times a day, five times a day or six times a day or more.
24. The use of any of the preceding forms, wherein the daily dosage of the medicament, or one ingredient of the medicament, is increased or “ramped up” every week, or every other week, by about 25, 50, 75, 100, 125, 150, 175, 200, 225, 250, 275, 300, 325, 350, 375, 400, 425, 450, 475, 1500, 2000, 2500, 3000, 3500, 4000, or more or more mg per week, or every other week,
and optionally this “ramping up” or increasing of dosages continues for about a month, about 6 months or about a year, or until symptoms of IBD significantly diminish or abate, or significantly diminish or abate without need for administration of the medicament.
25. The use of any of the preceding forms, wherein the medicament further comprises a flavoring or a sweetening agent, an aspartamine, a stevia, monk fruit, a sucralose, a saccharin, a cyclamate, a xylitol, a vanilla, an artificial vanilla or chocolate or strawberry flavor, an artificial chocolate essence, or a mixture or combination thereof.
26. The use of any of the preceding forms, wherein the medicament further comprises a preservative, a benzoic acid or a potassium sorbate.
27. The use of any of the preceding forms, wherein the medicament further comprises, or has added to: at least one probiotic or prebiotic,
wherein optionally the prebiotic comprises an inulin, lactulose, extracts of artichoke, chicory root, oats, barley, various legumes, garlic, kale, beans or flacks or an herb,
and optionally the probiotic comprises a cultured or stool-extracted microorganism or bacteria, or a bacterial component, and optionally the probiotic bacteria or bacterial component comprises or is derived from a non-pathogenic Clostridia, a Bacteroidetes, a Firmicutes, a Lactobacilli, a Bifidobacteria, an E. coli, a Strep fecalis, an Actinobacteria, a Proteobacteria, a Verruco-microbia, a Fusobacteria, a Cyanobacteria, a Spirochetes and a Lentisphaerae, and equivalents.
28. The use of any of the preceding forms, wherein the medicament further comprises, or has added to: at least one congealing agent, wherein optionally the congealing agent comprises an arrowroot or a plant starch, a powdered flour, a powdered potato or potato starch, an absorbant polymer, an Absorbable Modified Polymer, and/or a corn flour or a corn starch.
29. The use of any of the preceding forms, wherein the medicament further comprises an additive selected from one or more of a saline, a media, a defoaming agent, a surfactant agent, a lubricant, an acid neutralizer, a marker, a cell marker, a drug, an antibiotic, a contrast agent, a dispersal agent, a buffer or a buffering agent, a sweetening agent, a debittering agent, a flavoring agent, a pH stabilizer, an acidifying agent, a preservative, a desweetening agent and/or coloring agent, vitamin, mineral and/or dietary supplement, or a prebiotic nutrient.
30. The use of any of the preceding forms, wherein the medicament further comprises, or has added to: at least one Biofilm Disrupting Compound, wherein optionally the biofilm disrupting compound comprises an enzyme, a deoxyribonuclease (DNase), N-acetylcysteine, an auranofin, an alginate lyase, glycoside hydrolase dispersin B; a Quorum-sensing inhibitor, a ribonucleic acid III inhibiting peptide, Salvadora persica extracts, Competence-stimulating peptide, Patulin and penicillic acid; peptides—cathelicidin-derived peptides, small lytic peptide, PTP-7, Nitric oxide, neo-emulsions; ozone, lytic bacteriophages, lactoferrin, xylitol hydrogel, synthetic iron chelators, cranberry components, curcumin, silver nanoparticles, Acetyl-11-keto-β-boswellic acid (AKBA), barley coffee components, probiotics, sinefungin, S-adenosylmethionine, S-adenosyl-homocysteine, Delisea furanones, N-sulfonyl homoserine lactones or any combination thereof.
31. The use of any of the preceding forms, wherein the medicament is formulated as a delayed or gradual enteric release composition or formulation, and optionally the formulation comprises a gastro-resistant coating designed to dissolve at a pH of 7 in the terminal ileum, e.g., an active ingredient is coated with an acrylic based resin or equivalent, e.g., a poly(meth)acrylate, e.g. a methacrylic acid copolymer B, NF, which dissolves at pH 7 or greater, e.g., comprises a multimatrix (MMX) formulation.
32. The use of any of the preceding forms, wherein the medicament is contained in a delivery vehicle, product of manufacture, container, syringe, device or bag.
33. The use of any of the preceding forms, wherein the medicament is initially manufactured or formulated as a liquid, a suspension, a gel, a geltab, a semisolid, a tablet, a sachet, a lozenge or a capsule, or as an enteral formulation, or re-formulated for final delivery as a liquid, a suspension, a gel, a geltab, a semisolid, a tablet, a sachet, a lozenge or a capsule, or as an enteral formulation.

In a third aspect, the invention may include the following forms.

1. A pharmaceutical composition for use in treating, ameliorating, reversing, causing the remission of, and/or preventing (acting as a prophylaxis, or preventing the initiation of) an inflammatory bowel disease or disorder (IBD); ulcerative colitis; Crohn's disease; J-pouch; fistulising Crohn's disease; a colitis which can be microscopic, lymphocytic or collagenous; an eosinophilic colitis; indeterminate colitis; idiopathic colitis; diverticulosis; diverticulitis; relapsing diverticulitis; constipation associated inflammatory bowel disease and/or small intestinal bacterial overgrowth; irritable bowel syndrome (IBS) with or without diarrhoea, constipation or pain predominant IBS; periodontitis; rheumatoid arthritis; respiratory infections, appendicitis, vascular disorders such as thrombophlebitis; bacteremia; osteomyelitis; septic shock; Alzheimer's disease; Lemierre syndrome (postanginal sepsis); colonic polyps; or adenomas (optionally hyperplastic, adenomatous or serrated adenomas); or
preventing the growth of, or slowing the progression or recurrence of, colonic polyps or adenomas, bowl cancer, or metastases optionally preventing the initiation of or recurrence of or promotion of bowl cancer or metastasis; pharyngitis; otitis; or sinusitis; or
treating, ameliorating, reversing, causing the remission of, and/or preventing any disease, symptom or condition caused or exacerbated by a Fusobacteria infection, e.g., a F. nucleatum, F. varium, F. simae, F. periodonticum, F. equimun, or F. Necrogenes) infection,
in an individual in need thereof, the pharmaceutical composition comprising or consisting of:
(a) (i) a rifaximin (or, In-25-yl acetate; (16Z,18E,28E)-5,6,21,23-tetrahydroxy-27-methoxy-2,4,11,16,20,22,24,26-octamethyl-1,15-dioxo-1,2-dihydro-2,7-(epoxypentadeca[1,11,13]trienoimino)furo[2″,3″:7′,8′]naphtho[1′,2′:4,5]imidazo[1,2-a]pyrid; or, 2S-Acetyloxy-5,6,21,23-tetrahydroxy-27-methoxy-2,4,11, 16,20,22,24,26-octamethyl-2,7-(epoxypentoeleca(1,11,13)trienimino)benzofuro[4,5-e]pyride[1,2-a]benzimidazole-1,15(2H)-dione; 80621-81-4; or, enantiomers or stereoisomers thereof) (optionally a XIFAXAN™, XIFAXANTA™, RITACOL™, FATROXIMIN™, XIFAXSAN™, RIFAXIMINUM™, RIFAXIMINUN™, RIFAXIMINE™, RIFAXIMIN™, RIFAXIDIN™, RIFAXIMINA™, RIFAMYCIN™, VETRANAL™, or NORMIX™), a polymorphic form of a rifaximin or a rifaximin equivalent thereof, an extended intestinal release (EIR) rifaximin, a rifamycin derivative, a rifampicin (or rifampin) (optionally RIFADIN™), a rifabutin (optionally MYCOBUTIN™), a rifapentin (optionally PRIFTIN™), a rifalazil, a bicozamycin, a pyrido-imidazo rifamycin, a dehydro-rifaximin, a rifaximin histidine, a rifaximin tryptophan, an 11-desmethyl-rifaximin (e.g., an 11-desmethyl NORMIX™), a rifaximin beta-cyclodextrin, fosfomycin, or equivalents thereof or a mixture or a combination thereof,
(ii) vancomycin, neomycin, tobramycin, paromomycin or streptomycin,
(iii) gentamicin, streptomycin, ridinilazole, teicoplanin, streptomycin and neomycin, fidaxomicin, ramoplanin, surotomycin, capozide, a partially absorbed agent, optionally comprising a tinidazole, metronidazole, nitazoxanide, amoxicillin, tetracyclines, ornidazole, secnidazole, ciprofloxacin or an ansamycin, or
(iv) an antibiotic or drug as listed in Table 1; or
(b) an antibiotic or drug of (a), and at least one additional antimicrobial or antibiotic agent,
wherein optionally for (a) or (b) the rifaximin or rifaximin polymorphic form thereof or rifaximin equivalent comprises:
(i) a rifaximin, a rifaximin polymorph or a rifaximin equivalent as described in U.S. Pat. No. 9,273,066, optionally comprising a polymorphic form zeta of rifaximin exhibiting an X-ray powder diffraction pattern having characteristic peaks expressed in degrees 2 theta (+/−0.20 degree theta) comprising 4.69, 7.63, 12.52, 13.87;
(ii) a 25-desacetyl rifaximin, or a rifaximin, a rifaximin polymorph or a rifaximin equivalent, as described in U.S. Pat. No. 9,364,467, optionally comprising a 25-desacetyl rifaximin or a pharmaceutically acceptable salt thereof (optionally a sodium, potassium, calcium, magnesium, ammonium, or chlorine pharmaceutically acceptable salt of 25-desacetyl rifaximin), wherein optionally the 25-desacetyl rifaximin has the formula:

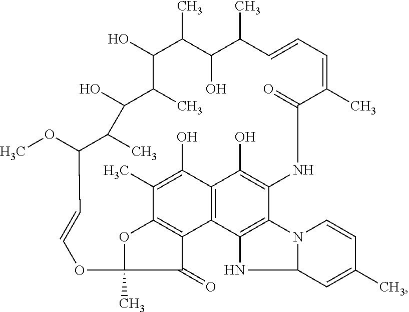 [SMILES: COC1/C=C/O[C@@]2(C)Oc3c(C)c(O)c4c(O)c(c5c(c4c3C2=O)NC2C=C(C)C=CN52)NC(=O)/C(C)=C\C=C\C(C)C(O)C(C)C(O)C(C)C(O)C1C]

(iii) a rifaximin, a rifaximin polymorph or a rifaximin equivalent as described in U.S. Pat. No. 9,546,183, optionally comprising a polymorphic Form B of rifaximin exhibiting an X-ray powder diffraction pattern having characteristic peaks expressed in degrees 2 theta (+/−0.20 degree theta) at 5.24, 6.84, 7.74, 8.71, 10.16, and 12.21,
(iv) a rifaximin amorphous form or a rifaximin equivalent as described in U.S. Pat. No. 9,700,545, optionally comprising an amorphous form of rifaximin exhibiting an X-ray powder diffraction pattern having characteristic peaks expressed in degrees: (1) 2 theta (+/−0.20 degree theta) at 7.3, 11.3-17.8, and 15.8 degrees 2 theta; 2 theta (+/−0.20 degree theta) at 5.1-10.1, 11.3-17.8, and 15.8 degrees 2 theta; or (3) 2 theta (+/−0.20 degree theta) at 5.1-10.1, 7.3, and 11.3-17.8 degrees 2 theta,
(v) a rifaximin, a rifaximin polymorph or a rifaximin equivalent as described in U.S. Pat. No. 9,359,374, or U.S. Pat. No. 9,725,466, optionally comprising a polymorphic form APO-III of rifaximin characterized by a PXRD diffractogram comprising peaks, in terms of degrees 2-theta, at approximately 7.1, 8.4, 11.6, 13.1, 18.5, 18.8, and 25.0,
(vi) a rifaximin, a rifaximin polymorph or a rifaximin equivalent as described in U.S. Pat. No. 9,421,195,
(vii) a rifaximin, a rifaximin polymorph or a rifaximin equivalent as described in U.S. Pat. No. 7,045,620, optionally a crystalline polymorphous form of a rifaximin, a rifaximin polymorph or a rifaximin equivalent; and/or
(viii) a controlled-release or spray-dried rifaximin, rifaximin polymorph or rifaximin equivalent as described in U.S. Pat. No. 9,498,442, optionally rifaximin, rifaximin polymorph or rifaximin equivalent characterized by an X-Ray diffraction spectrum showing diffraction halo peaks in the range 7.75 degree+−0.0.2-18.33 degree±0.0.2, 2 theta, with maximum at about 7.75 degree±0.0.2 and in the range 14.54 degree±0.0.2 and 18.33 degree±0.0.2, 2 theta.
2. The pharmaceutical composition for use of form 1, wherein the IBD further comprises or is associated with a condition or side effect comprising diarrhoea, rectal bleeding, mucus, urgency, incontinence, nocturnal diarrhoea; together with lower abdominal pain, weight loss, excessive gas production, bloating, loss of appetite, joint pains/symptoms, and optionally administration of the pharmaceutical composition treats, ameliorates, reverses, causes the remission of, and/or prevents (acts as a prophylaxis) one, several or all of these conditions or side effects.
3. The pharmaceutical composition for use of form 1 or form 2, further comprising at least one additional antimicrobial or antibiotic agent, or further comprises a drug or a probiotic.
4. The pharmaceutical composition for use of form 3, wherein the at least one additional antimicrobial or antibiotic agent comprises a vancomycin, a metronidazole (optionally FLAGYL™, METRO™), a tinidazole (optionally FASIGYN™, SIMPLOTAN™, TINDAMAX™), an ornidazole (optionally XYNOR™), a secnidazole (optionally FLAGENTYL™, SINDOSE™, SECNIL™), an antibiotic or drug as listed in Table 1, or a combination thereof.
5. The pharmaceutical composition for use of any of the preceding forms, wherein the at least one additional antimicrobial or antibiotic agent comprises: an antibiotic or antibacterial agent from one or more of the following classes selected from: tetracyclines, penicillins, macrolides, quinolones, chloramphenicol, rifamycins, sulphonamides, co-trimoxazole, and oxazolidinones.
6. The pharmaceutical composition for use of any of the preceding forms, wherein the at least one additional antimicrobial or antibiotic agent comprises: a doxycycline, chlortetracycline, tetracycline hydrochloride, oxytetracycline, demeclocycline, methacycline, minocycline, penicillin, amoxycillin, erythromycin, clarithromycin, roxithromycin, azithromycin, spiramycin, oleandomycin, josamycin, kitsamysin, flurithromycin, nalidixic acid, oxolinic acid, norfloxacin, perfloxacin, amifloxacin, ofloxacin, moxifloxacin, ciprofloxacin, sparfloxacin, levofloxacin, rifabutin, rifampicin, rifapentin, rifalazil, sulfisoxazole, sulfamethoxazole, sulfadiazine, sulfadoxine, sulfasalazine, sulfaphenazole, dapsone, sulfacytidine, linezolid or any combination thereof.
7. The pharmaceutical composition for use of any of the preceding forms, wherein the at least one additional antimicrobial or antibiotic agent comprises:
an ampicillin, a sulbactama tetracycline, a cephalosporin, a carbapenem, an imipenem, a meropenem, a monobactam, a lincosamide, a clindamycin, a quinolone, a fluoroquinolone, a sulphonamide, a fradicin, a nitroimidazole, a metronidazole, a tinidazole, a secnidazole, an anti-Clostridial agent, or a ramoplanan,
an aminoglycoside antibiotic, a gentamycin, a neomycin, a streptomycin, a paromomycin, a verdamicin, a mutamicin, a sisomicin, a netilmicin, a retymicin, a kanamycin, an amphenicol, an ansamycin, a beta-lactam (β-lactam) antibiotic, a carbapenem, a cephalosporin, a cephamycin, a monobactam, an oxacephem, a lincosamide antibiotic, a clindamycin, or a lincomycin,
a glycopeptide antibiotic, a vancomycin, a teicoplanin, a telavancin, a bleomycin, a ramoplanin, a decaplanin, a polypeptide antibiotic, an actinomycin, an actinomycin D, a bacitracin, a bacitracin, a tetracycline, a 2,4-diaminopyrimidine class antibiotic, a clavacin, a clairformin, a claviform, an expansine, a clavatin, an expansin, a gigantin, a leucopin, a patuline or a patulin), or
an equivalent thereof or a combination thereof.
8. The pharmaceutical composition for use of any of the preceding forms, wherein the at least one additional antimicrobial or antibiotic agent comprises:
(i) a rifaximin (optionally a XIFAXAN™, XIFAXANTA™ or NORMIX™), RITACOL™), FATROXIMIN™), XIFAXSAN™), RIFAXIMINUM™), RIFAXIMINUN™), RIFAXIMINE™), RIFAXIMIN™), RIFAXIDIN™), RIFAXIMINA™, RIFAMYCIN™, a polymorphic form of a rifaximin or a rifaximin equivalent thereof, an extended intestinal release (EIR) rifaximin, a rifamycin derivative, a rifampicin (or rifampin) (optionally RIFADIN™), a rifabutin (optionally MYCOBUTIN™), a rifapentin (optionally PRIFTIN™), a rifalazil, a bicozamycin, a pyrido-imidazo rifamycin, a dehydro-rifaximin, a rifaximin histidine, a rifaximin tryptophan, an 11-desmethyl-rifaximin (e.g., an 11-desmethyl NORMIX™), a rifaximin beta-cyclodextrin, or equivalents thereof or a mixture or a combination thereof,
(ii) vancomycin, neomycin, tobramycin, paromomycin or streptomycin,
(iii) gentamicin, streptomycin, ridinilazole, teicoplanin, streptomycin and neomycin, fidaxomicin, ramoplanin, surotomycin, capozide, a partially absorbed agent, optionally comprising a tinidazole, metronidazole, nitazoxanide, amoxicillin, tetracyclines, ornidazole, secnidazole, ciprofloxacin or an ansamycin, or
(iv) an antibiotic or drug as listed in Table 1, or
(v) any combination thereof.
9. The pharmaceutical composition for use of any of the preceding forms, wherein the antimicrobial or antibiotic agent comprises a three-drug therapeutic combination comprising:
rifaximin, tinidazole and nitazoxanide; rifaximin, tinidazole and oral tobramycin; rifaximin, amoxicillin and metronidazole; rifaximin, paromomycin and nitazoxanide; rifaximin, paromomycin and vancomycin; rifaximin, tinidazole and paromomycin; rifaximin, ridinilazole and tobramycin; rifaximin, ridinilazole and paromomycin; or rifaximin, ridinilazole and nitazoxanide; and optionally teicoplanin is substituted for any one of the second or third drug in this 3-drug combination list.
10. The pharmaceutical composition for use of any one of forms 1 to 8, comprising a rifamycin, a nitroimidazole, and a tetracycline antibiotic.
11. The pharmaceutical composition for use of form 10 wherein the rifamycin is rifampicin, the nitroimidazole is secnidazole, and the tetracycline antibiotic is doxycycline.
12. The pharmaceutical composition for use of any one of forms 1 to 8, comprising a rifamycin, a nitroimidazole, and a thiazolide.
13. The pharmaceutical composition for use of form 12, wherein the rifamycin is rifaximin, the nitroimidazole is tinidazole, and the thiazolide is nitazoxanide.
14. The pharmaceutical composition for use of any one of forms 1 to 8, comprising fosfomycin, a nitroimidazole, and a tetracycline antibiotic.
15. The pharmaceutical composition for use of form 14, wherein the nitroimidazole is metronidazole, and the tetracycline antibiotic is doxycycline.
16. The pharmaceutical composition for use of any of the preceding forms, wherein in said use the individual exhibits at least an about 5% to 10%, or 10% to 20%, or 20% to 30%, or 30% to 40%, or 40% to 50%, or 50% to 60%, or 70% to 80%, or 80% to 90%, or substantially complete, reduction in IBD symptoms or side effects or severity after administration of the pharmaceutical composition to the individual in need thereof as compared to before initiating the administration.
17. The pharmaceutical composition for use of form 16, wherein the at least an about 5% to 10%, or 10% to 20%, or 20% to 30%, or 30% to 40%, or 40% to 50%, or 50% to 60%, or 70% to 80%, or 80% to 90%, or substantially complete, reduction in IBD symptoms or side effects or severity is achieved after about 1, 2 or 3 or more weeks, or after about 1 to 2 months, or after about 2 to 6 months, of initiating the administration.
18. The pharmaceutical composition for use of form 16, wherein the at least an about 5% to 10%, or 10% to 20%, or 20% to 30%, or 30% to 40%, or 40% to 50%, or 50% to 60%, or 70% to 80%, or 80% to 90%, or substantially complete, reduction in IBD symptoms or side effects or severity is maintained for at least about 4 to 8 weeks, or 2 to 6 months, after discontinuing the administration to the individual.
19. The pharmaceutical composition for use of any of the preceding forms, formulated as a chewable delivery vehicle, a gum, a gummy, a candy, a lozenge, an ice cream or an ice, a yogurt or a drink.
20. The pharmaceutical composition for use of any of the preceding forms, wherein a unit dosage is a pediatric unit dosage, and optionally the unit dosage is between about 10 mg and 1100 mgm, or between about between about 40 mg and 4,000 mgm, or is about 10, 20, 30, 40, 50, 60, 70, 75, 80, 90, 100, 125, 150, 175, 200, 225, 250, 275, 300, 325, 350, 375, 400, 425, 450, 475, 500, 600, 700, 750, 800, 900, 1000, 1100, 1500, 2000, 2500, 3000, 3500, 4000, or more mg per unit dose, which optionally can be administered once a day, bid or tid, or a four times a day, five times a day or six times a day or more, regimen.
21. The pharmaceutical composition for use of any of the preceding forms, wherein a daily dosage is about 50, 75, 100, 125, 150, 175, 200, 225, 250, 275, 300, 325, 350, 375, 400, 425, 450, 475, 500, 600, 700, 750, 800, 900, 1000, 1100, 1500, 2000, 2500, 3000, 3500, 4000, or more mg per day, or between about 100 and 1100 mgm per day total, or between about 400 and 4000 mg per day, which optionally can be administered in a once a day, bid or tid, or four times a day, five times a day or six times a day or more, regimen.
22. The pharmaceutical composition for use of any of the preceding forms, wherein a unit dosage is set for (the daily dosage is set for) bid (twice a day), tid (three times a day), four times a day, five times a day or six times a day or more, with the unit dosage and daily dosage adjusted to be: about 1000 mg/70 kg a day, or about 14 mg/kg a day, for an adult median dose per day; or for a pediatric dosage about 350 mg/25 kg a day, or about 15 to 16 mg/kg, a day; or equivalent.
23. The pharmaceutical composition for use of any of the preceding forms, wherein the daily dosage is about 25 mg to 20 grams (gm) bid, or about 400, 425, 450, 475, 500, 600, 700, 750, 800, 900, 1000, 1100, 1200, 1500, 2000, 2500, 3000, 3500, 4000, or more mgm once a day, bid or tid, or four times a day, five times a day or six times a day or more.
24. The pharmaceutical composition for use of any of the preceding forms, wherein the daily dosage is, or wherein one ingredient of the pharmaceutical composition for use, is increased or “ramped up” every week, or every other week, by about 25, 50, 75, 100, 125, 150, 175, 200, 225, 250, 275, 300, 325, 350, 375, 400, 425, 450, 475, 1500, 2000, 2500, 3000, 3500, 4000, or more or more mg per week, or every other week,
and optionally this “ramping up” or increasing of dosages continues for about a month, about 6 months or about a year, or until symptoms of IBD significantly diminish or abate, or significantly diminish or abate without need for administration of the pharmaceutical composition.
25. The pharmaceutical composition for use of any of the preceding forms, further comprising a flavoring or a sweetening agent, an aspartamine, a stevia, monk fruit, a sucralose, a saccharin, a cyclamate, a xylitol, a vanilla, an artificial vanilla or chocolate or strawberry flavor, an artificial chocolate essence, or a mixture or combination thereof.
26. The pharmaceutical composition for use of any of the preceding forms, further comprising a preservative, a benzoic acid or a potassium sorbate.
27. The pharmaceutical composition for use of any of the preceding forms, further comprising at least one probiotic or prebiotic,
wherein optionally the prebiotic comprises an inulin, lactulose, extracts of artichoke, chicory root, oats, barley, various legumes, garlic, kale, beans or flacks or an herb,
and optionally the probiotic comprises a cultured or stool-extracted microorganism or bacteria, or a bacterial component, and optionally the probiotic bacteria or bacterial component comprises or is derived from a non-pathogenic Clostridia, a Bacteroidetes, a Firmicutes, a Lactobacilli, a Bifidobacteria, an E. coli, a Strep fecalis, an Actinobacteria, a Proteobacteria, a Verruco-microbia, a Fusobacteria, a Cyanobacteria, a Spirochetes and a Lentisphaerae, and equivalents.
28. The pharmaceutical composition for use of any of the preceding forms, further comprising at least one congealing agent, wherein optionally the congealing agent comprises an arrowroot or a plant starch, a powdered flour, a powdered potato or potato starch, an absorbant polymer, an Absorbable Modified Polymer, and/or a corn flour or a corn starch.
29. The pharmaceutical composition for use of any of the preceding forms, further comprising an additive selected from one or more of a saline, a media, a defoaming agent, a surfactant agent, a lubricant, an acid neutralizer, a marker, a cell marker, a drug, an antibiotic, a contrast agent, a dispersal agent, a buffer or a buffering agent, a sweetening agent, a debittering agent, a flavoring agent, a pH stabilizer, an acidifying agent, a preservative, a desweetening agent and/or coloring agent, vitamin, mineral and/or dietary supplement, or a prebiotic nutrient.
30. The pharmaceutical composition for use of any of the preceding forms, further comprising at least one Biofilm Disrupting Compound, wherein optionally the biofilm disrupting compound comprises an enzyme, a deoxyribonuclease (DNase), N-acetylcysteine, an auranofin, an alginate lyase, glycoside hydrolase dispersin B; a Quorum-sensing inhibitor, a ribonucleic acid III inhibiting peptide, Salvadora persica extracts, Competence-stimulating peptide, Patulin and penicillic acid; peptides—cathelicidin-derived peptides, small lytic peptide, PTP-7, Nitric oxide, neo-emulsions; ozone, lytic bacteriophages, lactoferrin, xylitol hydrogel, synthetic iron chelators, cranberry components, curcumin, silver nanoparticles, Acetyl-11-keto-β-boswellic acid (AKBA), barley coffee components, probiotics, sinefungin, S-adenosylmethionine, S-adenosyl-homocysteine, Delisea furanones, N-sulfonyl homoserine lactones or any combination thereof.
31. The pharmaceutical composition for use of any of the preceding forms, formulated as a delayed or gradual enteric release composition, and optionally the composition comprises a gastro-resistant coating designed to dissolve at a pH of 7 in the terminal ileum, e.g., an active ingredient is coated with an acrylic based resin or equivalent, e.g., a poly(meth)acrylate, e.g. a methacrylic acid copolymer B, NF, which dissolves at pH 7 or greater, e.g., comprises a multimatrix (MMX) formulation.
32. The pharmaceutical composition for use of any of the preceding forms, contained in a delivery vehicle, product of manufacture, container, syringe, device or bag.
33. The pharmaceutical composition for use of any of the preceding forms, initially manufactured or formulated as a liquid, a suspension, a gel, a geltab, a semisolid, a tablet, a sachet, a lozenge or a capsule, or as an enteral formulation, or re-formulated for final delivery as a liquid, a suspension, a gel, a geltab, a semisolid, a tablet, a sachet, a lozenge or a capsule, or as an enteral formulation.

### DESCRIPTION OF EMBODIMENTS

In alternative embodiments, provided are pharmaceutical compositions, therapeutic combinations, devices and methods for treating, ameliorating, reversing (e.g., causing or inducing the remission of) and/or preventing (acting as a prophylaxis) an inflammatory bowel disease or an inflammatory bowel disorder (both collectively referred to as IBD).

Pharmaceutical compositions, therapeutic combinations, devices and methods as provided herein, by treating, ameliorating or inducing remission of IBD, can reduce, prevent or abate the symptoms of IBD, including diarrhoea, rectal bleeding, mucus, urgency, incontinence, nocturnal diarrhoea; together with lower abdominal pain, weight loss, excessive gas production, bloating, loss of appetite, joint pains/symptoms; and without the benefit of pharmaceutical compositions, therapeutic combinations, devices and methods as provided herein, these symptoms otherwise would continue on unabated. Because IBD is thought to be caused by an aberrant reaction in genetically predisposed patients to human normal gut flora, the inflammatory response, including e.g., redness and contact bleeding in the bowel, is treated as an immune reaction, and so various forms of immune suppression are typically used—and these treatments cannot induce remission; however, pharmaceutical compositions, therapeutic combinations, devices and methods can solve the problem of induction of remission of IBD, which can be very difficult and in some patients never occurs, leading to the removing surgically of the colon and leaving the patient either with a stoma or with a J-pouch.

Given some of the antibiotic combinations provided herein target a Fusobacteria, e.g., F. nucleatum or F. varium, infection, which are known to promote various infective conditions such periodontitis, rheumatoid arthritis, respiratory infections, appendicitis, vascular disorders, Alzheimer's disease, colonic polyps or adenomas (optionally hyperplastic, adenomatous or serrated adenomas) or preventing the growth of, or slowing the progression or recurrence of, colonic polyps or adenomas and bowel cancer, and metastases, Lemierre syndrome (postanginal sepsis), pharyngitis, otitis and sinusitis, drug combinations as described herein also are applicable to prevent, ameliorate, treat and/or lessen the symptoms of such infections and conditions.

In addition to targeting IBD, Fusobacteria can stimulate the growth of colonic polyps, including hyperplastic, adenomatous and serrated adenomas, as well as the initiation of the growth of bowel cancer. Accordingly, provided herein are therapeutic compositions and therapies effective for stopping, slowing the progression or recurrence of, or preventing, the growth of polyps or adenomas, including preventing or slowing their growth, including preventing, inhibiting or slowing the growth of a bowel cancer. Hence, use of these exemplary embodiments can reduce the costs of colonoscopic surveillance, currently running at about 15,000,000 procedures per year in the US alone. Provided herein are pharmaceutical compositions, therapeutic combinations, devices and methods that comprise ‘triple therapy’, dual therapy or monotherapy. In alternative embodiments, the ‘triple therapy’ can best inhibit the growth of IBD-related microbiome pathogenic bacteria and can obtain a prolonged remission of IBD (including histological and clinical remission) in IBD patients, which can put the IBD patient into an immunosuppressant-free therapeutic pathway.

In alternative embodiments, provided are pharmaceutical compositions, therapeutic combinations, devices and methods comprising use of single or combined antimicrobial agents, including poorly absorbed and/or well absorbed components; and including use of drugs used where bacterial infective components are resistant or sensitive or development of resistance occurs; which in alternative embodiments have the aim or clinical goal of suppressing the luminal flora and treating the impenetrable mucosal layer over the mucosa or biofilm, thus accessing intracellular spaces where the pathogens may hide.

In alternative embodiments, provided are pharmaceutical compositions, therapeutic combinations, devices and methods comprising use of single or combined antimicrobial agents for the treatment of a pathogenic Fusobacterium bacterium such as a F. nucleatum or F. varium e.g., including its relationship to the appendix either with its removal or being left in place. With the advancement of microbial detection technologies, an increasing number of previously overlooked microorganisms have been discovered to play important roles in human diseases, including Fusobacterium nucleatum, a Gram-negative anaerobe, is such an emerging pathogen that is quickly attracting attention of the medical and research communities. F. nucleatum is ubiquitous in the oral cavity, absent or infrequently detected elsewhere in the body under normal conditions. Under disease conditions, however, F. nucleatum is one of the most prevalent species found in extra-oral sites. F. nucleatum is a heterogeneous species with five proposed subspecies (ss), i.e. ss animalis, ss fusiforme, ss nucleatum, ss polymorphum, and ss vincentii, whose prevalence in disease vary. Fusobacterium varium can also be similarly associated with IBD as can other Fusobacteria previously thought to be commensal.

In alternative embodiments, provided are pharmaceutical compositions, therapeutic combinations, devices and methods comprising use of: oral and/or enteric-coated or enema products; or, co-therapy with probiotics reflecting the human flora that could be cultured to help with a dysbiosis; cycling antibiotics with the probiotics; and/or, co-therapy with anti-inflammatory agents to accelerate inflammation resolution.

In alternative embodiments, pharmaceutical compositions, therapeutic combinations, devices and methods of use thereof as provided herein can effect (can result in, or cause) a prolonged, deep mucosal healing, including a histologic, visual and clinical remission of an IBD.

In alternative embodiments, provided are pharmaceutical compositions, therapeutic combinations, devices and methods for treating, ameliorating, reversing, causing the remission of, and/or preventing (acting as a prophylaxis, or preventing the initiation of) an inflammatory bowel disorder (IBD) or inflammatory bowel disease (IBD) in an individual in need thereof, comprising administering to the individual in need thereof a formulation, a pharmaceutical preparation, a therapeutic combination, or a pharmaceutical composition comprising or consisting of: (a) (i) a rifaximin (optionally a XIFAXAN™, XIFAXANTA™, RITACOL™), FATROXIMIN™), XIFAXSAN™), RIFAXIMINUM™), RIFAXIMINUN™), RIFAXIMINE™), RIFAXIMIN™), RIFAXIDIN™), RIFAXIMINA™, RIFAMYCIN™, or NORMIX™), a polymorphic form of a rifaximin or a rifaximin equivalent thereof, an extended intestinal release (EIR) rifaximin, a rifamycin derivative, a rifampicin (or rifampin) (optionally RIFADIN™), a rifabutin (optionally MYCOBUTIN™), a rifapentin (optionally PRIFTIN™), a rifalazil, a bicozamycin, a pyrido-imidazo rifamycin, a dehydro-rifaximin, a rifaximin histidine, a rifaximin tryptophan, an 11-desmethyl-rifaximin (e.g., an 11-desmethyl NORMIX™), a rifaximin beta-cyclodextrin, or equivalents thereof or a mixture or a combination thereof, or an antibiotic or drug as listed in Table 1; or, (b) an antibiotic or drug of (a), and at least one additional antimicrobial or antibiotic agent.

TABLE 1

| Antibiotic Classes | Generic name (Brand Name) |
|---|---|
| Aminoglycosides | Amikacin (Amikin), Gentamicin (Garamycin), Kanamycin |
| | (Kantrex), Neomycin (Neo-Fradin), Netilmicin (Netromycin), |
| | Tobramycin (Nebcin), Paromomycin (Humatin), Streptomycin |
| | (N/A), Spectinomycin (Trobicin) |
| Ansamycins | Geldanamycin (Trastuzumab), Herbimycin (N/A), Rifaximin |
| | (Xifaxan),Rifabutin (Mycobutin), Rifampicin (Rifampin), Rifalazil, |
| | Rifapentine; Tanespimycin |
| Carbacephem | Loracarbef (Lorabid) |
| Carbapenems | Ertapenem (Invanz), Doripenem (Doribax), Imipenem/Cilastatin |
| | (Primaxin), Meropenem (Merrem) |
| Cephalosporins | Cefadroxil (Duricef), Cefazolin (Ancef), Cefalexin (Keflex) |
| (First generation) | |
| Cephalosporins | Cefaclor (Distaclor), Cefprozil (Cefzil), Cefuroxime (Ceftin, |
| (Second generation) | Zinnat) |
| Cephalosporins | Cefixime (Cefspan), Cefdinir (Omnicef, Cefdiel), Cefditoren |
| (Third generation) | (Spectracef, Meiact), Cefoperazone (Cefobid), Cefotaxime |
| | (Claforan), Cefpodoxime (Vantin, Banadoz), Ceftazidime (Fortaz), |
| | Ceftibuten (Cedax), Ceftriaxone (Rocephin) |
| Cephalosporins | Cefepime (Maxipime) |
| (Fourth generation) | |
| Cephalosporins | Ceftaroline fosamil (Teflaro), Ceftobiprole (Zeftera) |
| (Fifth generation) | |
| Glycopeptides | Teicoplanin (Targocid), Vancomycin (Vancocin),Telavancin |
| | (Vibativ), Dalbavancin (Dalvance), Oritavancin (Orbactiv) |
| Lincosamides | Clindamycin (e.g., CLEOCIN ™, DALACIN ™, CLINACIN ™), |
| | Lincomycin (Lincocin) |
| Lipopeptide | Daptomycin (Cubicin) |
| Macrolides | Azithromycin (Zithromax, Surnamed, Xithrone), Clarithromycin |
| | (Biaxin), Erythromycin (Erythocin, Erythroped), Roxithromycin |
| | (N/A), Telithromycin (Ketek), Spiramycin (Rovamycine) |
| Monobactams | Aztreonam (Azactam) |
| Nitrofurans | Furazolidone (Furoxone), Nitrofurantoin (Macrodantin, Macrobid) |
| Nitroimidazoles | Tinidazole (Fasigyn, Simplotan, Tindamax), Metronidazole |
| | (Flagyl), Ornidazole (Ornigil), Secnidazole |
| Oxazolidinones | Linezolid (Zyvox), Posizolid (N/A), Radezolid (N/A), Torezolid |
| | (Sivextro); Cadazolid |
| Penicillins | Amoxicillin (Novamox, Amoxil), Ampicillin (Principen), |
| | Azlocillin, Dicloxacillin (Dynapen), Flucloxacillin (Floxapen), |
| | Mezlocillin (Mezlin), Methicillin (Staphcillin), Nafcillin (Unipen), |
| | Oxacillin (Prostaphlin), Penicillin G (Pentids), Penicillin V |
| | (Veetids), Piperacillin (Pipracil), Penicillin G (Pfizerpen), |
| | Temocillin (Negaban), Ticarcillin (Ticar) |
| Penicillin | Amoxicillin/clavulanate (Augmentin), Ampicillin/sulbactam |
| combinations | (Unasyn), Piperacillin/tazobactam (Zosyn), Ticarcillin/clavulanate |
| | (Timentin) |
| Polypeptides | Bacitracin (Baciguent), Colistin (Coly-Mycin-S), Polymyxin B |
| Quinolones/ | Ciprofloxacin (Cipro, Ciproxin, Ciprobay), Enoxacin (Penetrex), |
| Fluoroquinolones | Gatifloxacin (Tequin), Gemifloxacin (Factive), Levofloxacin |
| | (Levaquin), Lomefloxacin (Maxaquin), Moxifloxacin (Avelox), |
| | Nadifloxacin (Nadoxin), Nalidixic acid (NegGram), Norfloxacin |
| | (Noroxin), Ofloxacin (Floxin, Ocuflox), Trovafloxacin (Trovan), |
| | Grepafloxacin (Raxar), Sparfloxacin (Zagam), Temafloxacin |
| | (Omniflox) |
| Sulfonamides | Mafenide (Sulfamylon), Sulfacetamide (Sulamyd, Bleph-10), |
| | Sulfadiazine (Micro-Sulfon), Silver sulfadiazine (Silvadene), |
| | Sulfadimethoxine (Di-Methox, Albon), Sulfamethizole (Thiosulfil |
| | Forte), Sulfamethoxazole (Gantanol), Sulfanilimide (N/A), |
| | Sulfasalazine (Azulfidine), Sulfisoxazole (Gantrisin), Trimethoprim |
| | (Bactrim, Septra), Sulfamethoxazole (Gantanol), |
| | Sulfonamidochrysoidine (Prontosil) |
| Tetracyclines | Demeclocycline (Declomycin), Doxycycline (Vibramycin), |
| | Metacycline |
| | Minocycline (Minocin), Oxytetracycline (Terramycin), |
| | Tetracycline (Sumycin, Achromycin V, Steclin) |
| Drugs against | Clofazimine (Lamprene), Dapsone (Avlosulfon), Capreomycin |
| mycobacteria | (Capastat), Cycloserine (Seromycin), Ethambutol (Myambutol), |
| | Ethionamide (Trecator), Isoniazid (Nydrazid), Pyrazinamide |
| | (Aldinamide), Rifampicin (Rifadin, Rimactane), Rifabutin |
| | (Mycobutin), Rifapentine (Priftin), Streptomycin (N/A) |
| Others | Arsphenamine (Salvarsan), Chloramphenicol (Chloromycetin), |
| | Fosfomycin (Monurol, Monuril), Fusidic acid (N/A), |
| | Metronidazole (Flagyl), Mupirocin (B actroban), Platensimycin |
| | (N/A), Quinupristin/Dalfopristin (Synercid), Thiamphenicol, |
| | Tigecycline (Tigacyl), Tinidazole (Tindamax Fasigyn), |
| | Trimethoprim (Proloprim, Trimpex); Fidaxomicin (Marocyclic |
| | antibiotic-Dificid); Ridinilazole; Ramoplanin; Nitazoxanide; |
| | Tizoxanide; Surotomycin; |

N/A: Not available

In alternative embodiments, rifaximin (or a polymorphic form of a rifaximin or a rifaximin equivalent thereof, or an extended intestinal release (EIR) rifaximin, a rifamycin derivative, a rifampicin (or rifampin), a rifabutin, a rifapentin, a rifalazil, a bicozamycin, or a pyrido-imidazo rifamycin, a dehydro-rifaximin, a rifaximin histidine, a rifaximin tryptophan, an 11-desmethyl-rifaximin (e.g., an 11-desmethyl NORMIX™), a rifaximin beta-cyclodextrin, or equivalents thereof) alone, or in combination with other antibiotics or drugs, is used in a formulation, a pharmaceutical preparation, a therapeutic combination, or a pharmaceutical composition as provided herein, or to practice a method as provided herein. In alternative embodiments, rifaximin (or a polymorphic form of a rifaximin or a rifaximin equivalent thereof, or an extended intestinal release (EIR) rifaximin, a rifamycin derivative, a rifampicin (or rifampin), a rifabutin, a rifapentin, a rifalazil, a bicozamycin, or a pyrido-imidazo rifamycin, or equivalents thereof), alone, or in combination with other antibiotics or drugs, is formulated or administered, optionally in a ramping-up dose regimen, once a day, twice a day, three times a day or four times a day. In alternative embodiments, rifaximin (or a polymorphic form of a rifaximin or a rifaximin equivalent thereof, or an extended intestinal release (EIR) rifaximin, a rifamycin derivative, a rifampicin (or rifampin), a rifabutin, a rifapentin, a rifalazil, a bicozamycin, or a pyrido-imidazo rifamycin, or equivalents thereof), alone, or in combination with other antibiotics or drugs, is formulated or administered in a ramping-up dose regimen, optionally reaching a higher oral dose on a daily basis that has not been used before in clinical medicine; for example, in one embodiment, the rifaximin (or a polymorphic form of a rifaximin or a rifaximin equivalent thereof, or an extended intestinal release (EIR) rifaximin, a rifamycin derivative, a rifampicin (or rifampin), a rifabutin, a rifapentin, a rifalazil, a bicozamycin, or a pyrido-imidazo rifamycin, or equivalents thereof), alone, or in combination with other antibiotics or drugs, is started from about 550 mg twice daily (bid) (or between about 500 mgm and 1000 mgm bid), or about 550 mg three times daily (tid) (or between about 200 mgm and 1500 mgm tid, or 0.9, 1, 1.1 or 1.2 gm tid), or about 1.1 g twice daily (or between about 1 gm and 1.5 gm bid), optionally at 1.1 gram (g) three times per day to reach a maximum of about 6.6 g per day.

Surprisingly, it was found that a higher dose of rifaximin (or a polymorphic form of a rifaximin or a rifaximin equivalent thereof, or an extended intestinal release (EIR) rifaximin, a rifamycin derivative, a rifampicin (or rifampin), a rifabutin, a rifapentin, a rifalazil, a bicozamycin, or a pyrido-imidazo rifamycin, or equivalents thereof), as described above, spread over 3 doses/day (but in alternative embodiments, ×4, ×5, or more/day can be used in cases that are recalcitrant or unresponsive to previous treatments) works far better in inhibiting inflammation by inhibiting the infection(s) than the current recommended doses. Current doses are almost always an under-dosing with rifaximin; and there is no external dose reference or independent dose ranging study in colitis to be guided by. From the inventor's clinical experience, use of rifaximin alone can be used to achieve remission but only using the newly described higher and more frequent dosing as provided herein. Judging by how well rifaximin works at 3.3 g per day, it appears that the 550 mg twice daily has certainly been too low a dose to have been released on the market. Hence, provided herein is a more efficacious (as a significantly higher) dose of rifaximin (or a polymorphic form of a rifaximin or a rifaximin equivalent thereof, or an extended intestinal release (EIR) rifaximin, a rifamycin derivative, a rifampicin (or rifampin), a rifabutin, a rifapentin, a rifalazil, a bicozamycin, or a pyrido-imidazo rifamycin, or equivalents thereof) for induction of remission, or the treatment or amelioration thereof, of inflammatory bowel disease (IBD), thus providing higher frequencies of IBD remission and successful treatments.

In alternative embodiments, rifaximin (or a polymorphic form of a rifaximin or a rifaximin equivalent thereof, or an extended intestinal release (EIR) rifaximin, a rifamycin derivative, a rifampicin (or rifampin), a rifabutin, a rifapentin, a rifalazil, a bicozamycin, or a pyrido-imidazo rifamycin, or equivalents thereof) doses to be administered are about 3.3 g (grams) (or between about 3 to 4 g, or 2.5 to 4.5 g) twice daily (or dosaging up to about 6 to 9 g per day); and in alternative embodiments, this dosage (up to about 6 to 9 g per day) can be reached by ramping up the dosage (optionally, a slow ramping up) from an initial lower dosaging, which can be at about 550 mg bid. In alternative embodiments, this high dose treatment regimen can last weeks or months (up to 2, 3, 4, 5, 6, 7 months or more or for a year or more); noting that, because it is barely absorbed, rifaximin even at higher doses remains a very safe drug to take long-term.

In alternative embodiments, while the invention is not limited by any particular mechanism of action, a physiologic basis for the efficacy of the higher dosages as provided herein (a much higher dose then what is used now) is that known bacterial resistance development will be minimised.

Rifaximin even at higher doses is a very safe drug to take long-term, possibly because it is not absorbed from the gut. For example, analogously, a non-absorbed drug that is taken on a daily basis at high dosages by thousands of people around the world is polyethylene glycol (PEG) 3350, marketed as MOVICOL®, taken for constipation; where thousands of patients take at least 1 sachet of MOVICOL® which contains about 13 grams of PEG, and many take 3 sachets per day (about 39 g of PEG daily) on a long-term basis. When this is compared to the 1.1 g of rifaximin, which like PEG is also not significantly absorbed from the gut, the rifaximin dose is low in comparison to the 39 g of PEG taken daily on a long-term basis. Thus, high dosages as provided herein are safe to take and can prevent rifaximin-induced resistance (a reason being, e.g., while the invention is not limited by any particular mechanism of action, because the bacteria are so overwhelmed by the high administered dose bacterial drug resistance is prevented; also, higher dosages result in a better penetration of the mucus layer). A low rifaximin dose has previously been used for a short duration. e.g., for two weeks; however, with a drug that is minimally or not absorbed such as PEG or rifaximin, they are not restricted to two weeks or low dosage usage.

In alternative embodiments, an IBD or colitis minimum duration of therapy is about ten to twelve weeks, or about 8 to 11, 12, 13, 14 to 15 or more weeks, or between about 2 to 6 months, or until the patient reaches histological normality. In alternative embodiments, a sixteen, 17, 18, 19, 20 or more weeks treatment duration benefits the more severe IBD or colitis patients to achieve deeper remission in this chronic inflammatory bowel disease—a devastating condition which can lead even in young people up to 29% colectomy rate.

In alternative embodiments, also provided are methods comprising use of a formulation, a pharmaceutical preparation, a therapeutic combination, or a pharmaceutical composition as provided herein, e.g., using rifaximin alone or in combination with another antibiotic or drug, comprising use of a standard treatment dose of about 550 mg of rifaximin (or a polymorphic form of a rifaximin or a rifaximin equivalent thereof, or an extended intestinal release (EIR) rifaximin, a rifamycin derivative, a rifampicin (or rifampin), a rifabutin, a rifapentin, a rifalazil, a bicozamycin, or a pyrido-imidazo rifamycin, or equivalents thereof) three times daily (tid) as a background dose to produce stool levels that resemble a ‘sine wave’ in the gut; and to this is added an extended release dose to be taken simultaneously with the standard treatment dose with the goal of filling out any troughs in the sine wave; and while the invention is not limited by any particular mechanism of action, this dual dosage regimen (standard and extended release dosaging and/or formulating) does not allow the bacteria to be left without surrounding antibiotic (e.g., the rifaximin), and this works better to suppress rogue bacteria. This may reduce the cost for the patient by taking a lesser gram total of rifaximin or equivalent.

In alternative embodiments, also provided are methods and formulations, pharmaceutical preparations, therapeutic combinations or pharmaceutical compositions comprising use of orally administered medications that are virtually or substantially unabsorbed, for example, comprising use of poorly absorbed drugs such as rifaximin, vancomycin, neomycin and tobramycin and the like (which are particularly poorly absorbed during food intake), paromomycin, streptomycin and numerous other “mycin drugs”. As they are poorly absorbed they reach the colon as ingested and can have greater effects upon the gut flora. Thus, in alternative embodiments, rifaximin (or a polymorphic form of a rifaximin or a rifaximin equivalent thereof, or an extended intestinal release (EIR) rifaximin, a rifamycin derivative, a rifampicin (or rifampin), a rifabutin, a rifapentin, a rifalazil, a bicozamycin, or a pyrido-imidazo rifamycin, or equivalents thereof), vancomycin, neomycin, tobramycin, paromomycin, streptomycin and other aminoglycosides or “mycin drugs” (see Table 1) are combined with various agents that are either absorbed or poorly absorbed to create powerful suppression of the pathogens that would be causing various diseases e.g. inflammatory bowel disease (IBD), polyp growth, bowel cancer, appendicitis, and other Fusobacteria-related, e.g., F. nucleatum- or F. varium-related infections or conditions, e.g., as described herein.

In alternative embodiments, use of poorly absorbed drugs, as provided by methods and compositions as provided herein, solves the problem of using well-absorbed drugs, which can be re-secreted into the colon, where the absorption creates a possibility of adverse effects and metabolic imbalance as well as toxicity to various active anatomical structures e.g. metronidazole neuropathy with very long-term usage.

In alternative embodiments, use of high dosage and therapeutic drug combinations as used in formulations and administration regimens of the methods and compositions as provided herein address the problem of a possible development of antibiotic resistance. In alternative embodiments, resistance is developed less frequently by use of the various multiple antibiotic combinations as provided herein, e.g., there antibiotics or drugs are used simultaneously to prevent an antibiotic-resistant mutation from occurring; also, because mutations typically need to occur in three or four places simultaneously to overcome the resistance to a three or four different component mix of drugs, the occurrence of drug resistance is avoided.

In alternative embodiments, antibiotics effective against a Fusobacterium, e.g., a F. nucleatum or F. varium, infection, are used in methods and compositions as provided herein. Alternative aims when delivering antimicrobial agents to the colon is: to suppress the luminal flora content of the pathogens; suppress as much as possible the impenetrable mucosal layer lining the human tissue, i.e., the biofilm area; to enter (drugs to penetrate) into an inflamed colon tissue deeply as possible, e.g., where there is little mucus left and impact the intra- and inter-cellular spaces where pathogens are known to exist. Furthermore, in idiopathic inflammatory bowel disease (IBD) there is evidence that Fusobacteria may be capable of causing inflammation when administered to mice, and probably do the same in humans. Furthermore, anti-Fusobacterium and other antimicrobial agents can inhibit these bacteria and bring the patient's bowel inflammation into remission, sometimes even very prolonged remission. Since Fusobacterium reside in the appendix and cause appendicitis, the source in the appendix if removed before the age of twenty, largely prevents the subsequent development of ulcerative colitis. This points to the fact Fusobacterium are somehow related to the causality of colitis. Therefore, in alternative embodiments, a therapeutic option is to use a combination of antibiotics or antimicrobials that include drugs able to suppress or cure Fusobacterium infection in the lumen and the impenetrable mucus (for example, including clindamycin (e.g., CLEOCIN™, DALACIN™, CLINACIN™), and optionally also have the patients undergo an appendectomy prior to starting the antibiotics to remove the reservoir that may cause the relapse of the ulcerative colitis when the drugs are stopped.

In alternative embodiments, provided are pharmaceutical compositions and therapeutic combinations comprising one, two, three or several of the antibiotics and drugs listed in Table 1, for example: rifaximin, vancomycin, tobramycin, gentamicin, streptomycin, paromomycin, ridinilazole, teicoplanin, streptomycin and neomycin, fidaxomicin, ramoplanin, surotomycin, and/or capozide, also including the partially absorbed agents tinidazole, metronidazole, nitazoxanide, amoxicillin, tetracyclines, ornidazole, secnidazole, ciprofloxacin and other ansamycins such as rifampicin, rifabutin, and rifalazil.

In alternative embodiments, a pharmaceutical composition or therapeutic combination as provided herein comprises a double or triple drug combination, for example: rifaximin, Tobramycin or Rifaximin and Tinidazole; rifaximin and Metronidazole; or, any of the so-called “-mycins” with one of the non-mycin group.

In alternative embodiments, a pharmaceutical composition or therapeutic combination as provided herein comprises a two-drug combination, for example: rifaximin and a nitroimidazole such as metronidazole, tinidazole, nimorazole, dimetridazole, pretomanid, ornidazole, megazol, azanidazole or benznidazole; rifaximin and tinidazole; rifaximin and metronidazole; rifaximin and secnidazole; rifaximin and ornidazole; or alternatively, vancomycin, fidaxomycin, surotomycin and/or ridinilazole are substituted in the place of rifaximin in any one of these twin combinations, for example, vancomycin, fidaxomycin, surotomycin and/or ridinilazole with a nitroimidazole, e.g., a nitroimidazole as listed above.

In alternative embodiments, a pharmaceutical composition or therapeutic combination as provided herein comprises a two-drug combination, for example: rifaximin, vancomycin, fidaxomycin, surotomycin and/or ridinilazole with a rifampicin, nitazoxanide, tizoxanide, tobramycin, gentamycin or streptomycin.

In alternative embodiments, a pharmaceutical composition or therapeutic combination as provided herein comprises a two-drug combination, for example: amoxicillin and a nitroimidazole; rifaximin and amoxicillin; rifaximin and a tetracycline (e.g. doxycycline or tetracycline or hydrochloride); tobramycin and a tetracycline, tobramycin or rifaximin; tobramycin and amoxicillin; ciprofloxacin or levofloxacin with amoxicillin, metronidazole, tinidazole or a tetracycline; nitazoxanide with metronidazole, tinidazole, ornidazole or secnidazole; nitazoxanide and amoxicillin, rifampicin, rifaximin or rifabutin; nitazoxanide with a tetracycline; or, tobramycin with ciprofloxacin or levofloxacin and/or one of the ansamycins (e.g., a polyketide such as Azithromycin, Clarithromycin, Erythromycin, Fidaxomicin or Telithromycin).

In alternative embodiments, a pharmaceutical composition or therapeutic combination as provided herein comprises a three-drug combination, for example to cause a greater inhibition of biofilm, luminal and tissue intracellular and intercellular bacteria. As with tuberculosis, Helicobacter or Crohn's disease associated with Mycobacteria, dual therapy can be inadequate, and one has to practice triple therapy to inhibit development of resistance.

In alternative embodiments, a pharmaceutical composition or therapeutic combination as provided herein comprises a three-drug combination, for example: three of any of the antibiotics or drugs as listed in Table 1, e.g., exemplary 3-drug therapeutic combinations as provided herein comprise: rifaximin, tinidazole and nitazoxanide; rifaximin, tinidazole and oral tobramycin; rifaximin, amoxicillin and metronidazole; rifaximin, paromomycin and nitazoxanide; rifaximin, paromomycin and vancomycin; rifaximin, tinidazole and paromomycin; rifaximin, ridinilazole and tobramycin; rifaximin, ridinilazole and paromomycin; rifaximin, ridinilazole and nitazoxanide. In one embodiment, teicoplanin is substituted for any of the second or third drug in this 3-drug combination list, and optionally this provides for greater eradication of Fusobacteria, which are likely to be playing a role in the aetiology of inflammatory bowel disease (IBD).

In alternative embodiments, a pharmaceutical composition or therapeutic combination as provided herein comprises a four-drug combination, including e.g., any combination of drugs or antibiotics as listed in Table 1. In alternative embodiments, a 4-drug combination as provided herein is administered by cycling with two weeks on and two weeks off, particularly in those individuals in whom resistance to antibiotics appears to have taken place.

In alternative embodiments, a pharmaceutical composition or therapeutic combination as provided herein (including any one, two, three or four antibiotic combination) is used together with an immune modulating drug such as 6-mercaptopurine, methotrexate, azathioprine, an anti-TNF alpha drug (e.g., infliximab, or REMICADE™), ustekinumab (e.g., STELARA™) or thioguanine which can also be used as a suppository enema or as an orally administered thioguanine.

In alternative embodiments, a pharmaceutical composition or therapeutic combination as provided herein (including any one, two, three or four antibiotic combination) is used together with an anti-inflammatory medication such as 5-ASA compounds, prednisone, mesalazine or an anti-TNF agent.

In alternative embodiments, a pharmaceutical composition or therapeutic combination as provided herein (including any one, two, three or four antibiotic combination) is used together with an immunosuppressant, e.g., an anti-TNF agent such as infliximab, or REMICADE™, Humira, Cimzia, Simponi and Biosimilars; anti-integrins as Entyvio, Tysabri, Etrolizumab, Stelara, Risankizumab or Brazikumab. In alternative embodiments, a pharmaceutical composition or therapeutic combination as provided herein is used together a biologic, optionally administered orally, such as Otelza, and/or Jak inhibitors such as Xeljanz, Upadacitinib, Filgotinib, Ozanimod, Etrsimod.

In alternative embodiments, a pharmaceutical composition or therapeutic combination as provided herein (including any one, two, three or four antibiotic combination) is used together with an anti-CMV (cytomegalovirus) agent, or an anti-C. difficile agent such as vancomycin and metronidazole, anti-cryptosporidium, anti-Bacteroides, and anti-E. coli agents.

In alternative embodiments, an amoxil, tetracycline and metronidazole therapeutic combination as provided herein further comprises a rifaximin; or, amoxil, fosfomycin and metronidazole combination, optionally further comprising ongoing use of amoxicillin.

In alternative embodiments, in those patients who have co-existing Crohn's disease (CD), and are undergoing a therapy for CD, antimycobacterial treatment with rifabutin, clarithromycin and clofazimine is combined with rifaximin to accelerate the development of remission.

In alternative embodiments, a formulation, a pharmaceutical preparation, a therapeutic combination, or a pharmaceutical composition as provided herein, and any method as provided herein, is used for treating, ameliorating, reversing, causing the remission of, and/or preventing (acting as a prophylaxis, or preventing the initiation of): Ulcerative Colitis; Crohn's disease; J-pouch; fistulising Crohn's disease; a Colitis which can be microscopic, lymphocytic or collagenous; an eosinophilic colitis; indeterminate colitis; idiopathic colitis; diverticulosis and diverticulitis; relapsing diverticulitis; constipation associated inflammatory bowel disease and/or small intestinal bacterial overgrowth; Irritable Bowel Syndrome (IBS) with or without diarrhoea, constipation or pain predominant IBS.

### Probiotics

In alternative embodiments, in practicing methods and compositions as provided herein, co-therapies including (or further comprising) probiotics also can be used; the probiotics can be cultured to affect Fusobacteria and other co-existing pathogens. For example, in alternative embodiments, an antibiotic or a therapeutic combination as provided herein (e.g., rifaximin alone or with another drug) is combined with a probiotic such as Faecalibacterium prausnitzii, and this combination can be used in a trough situation (discussed above) to allow the Faecalibacterium prausnitzii to reach the target. The probiotic and/or antibiotic or therapeutic combination as provided herein can be enteric coated (separately or together) to better reach the distal small bowel and allow the drugs or antibiotics to become available in the colon. In alternative embodiments, numerous probiotics are used from various phyla e.g., Firmicutes, Bacteroidetes, Actinobacteria, Proteobacteria, Verruco-microbia, Fusobacteria, Cyanobacteria, Spirochetes and Lentisphaerae, from Archaea, fungi and viruses—so many types of probiotics and mixtures thereof can be employed with or after pre-treating luminal antibiotics described here. Non-pathogenic Clostridia and other Firmicutes as well as Bacteroides can also be used. In alternative embodiments, the probiotics can be vegetative form or in spore forms (particularly in situations where a spore form has an advantage because they are not affected by antibiotics or co-therapy with antibiotics).

In alternative embodiments, anti-inflammatory agents are used (or are administered) with spore-forming probiotics (optionally, the anti-inflammatory agent used first, then the spore-forming probiotic) to colonize the gut, e.g., with healthy Clostridia or Bacillus; this exemplary combination treatment is effective for IBD. In alternative embodiments, anti-inflammatory agents such as an aminosalicylate, e.g., 5-ASA (e.g., aspirin), steroids, anti-TNF alpha agents (e.g., infliximab (Remicade), adalimumab (Humira), certolizumab pegol (Cimzia), golimumab (Simponi), etanercept (Enbrel), thalidomide (Immunoprin), lenalidomide (Revlimid) and pomalidomide (Pomalyst, Imnovid)), and thiopurines (e.g., azathioprine, 6-mercaptopurine, and thioguanine), are used, optionally given by an oral route or by enteric coated medications. In alternative embodiments, these therapeutic combinations are used to accelerate suppression of the inflammatory process, thus shortening the treatment time, and optionally allowing use of a higher dose, which then can be reduced to a maintenance dose long-term. In alternative embodiments, these therapeutic combinations provide a deep mucosal healing, which provides mucosal histological normalisation accompanying clinical normalisation with normal calprotectin levels, e.g., in patients on maintenance antimicrobial agents or maintenance probiotics after the antimicrobial agents.

### Routes of Administration and Formulations

In alternative embodiments, in practicing methods and compositions as provided herein, a route of administration can be either oral, using tablets, oral enteric coated tablets, oral tablets that are non-extended released tablets or extended released tablets, and these can be combined with the non-extended release medications to cover the trough of the levels inside the stool. The medication can also be given as an enema which delivers a higher concentration to the colon where the absorption is minimal when compared with the small bowel.

In alternative embodiments, antibiotics and antibacterials used to practice pharmaceutical compositions, therapeutic combinations, devices and methods as provided herein are formulated and dosaged for oral administration as a powder, e.g., a lyophilised powder, which can be inserted into carriers, e.g., capsules, tablets, geltabs, and the like, e.g., for administration to autistic infants or children (or those suspected of developing an IBD) to ingest.

Because an IBD may present itself at a younger age or in disabled patients, children or some patients may find it difficult to swallow a capsule; thus, also provided are additional delivery vehicles, products of manufacture and devices to be combined with pharmaceutical compositions or therapeutic combinations as provided herein, e.g., powders such as lyophilised powders, e.g., lyophilised powder in a storage vehicle, e.g., capsules, lozenges, geltabs and the like; for example, provided are delivery vehicles, products of manufacture and devices manufactured as a container, a kit, a package or a pack of a “device and capsule” together, e.g., operably associated such that the container, kit, package or a pack permits individuals, e.g., the very young children and the older children (and including disabled or handicapped individuals) to ingest the product, e.g., the lyophilised product, from the storage vehicle, e.g., capsules, lozenges, geltabs and the like.

In alternative embodiments, the container, kit, a package or a pack provides the ability of any age child (or disabled or handicapped individual, or any individual) to ingest or swallow the product (e.g., a formulation, pharmaceutical preparation or pharmaceutical composition as provided herein) within the storage vehicle (e.g., capsule) by “draining”, e.g., by puncturing, crushing, twisting or turning the container by hand or a device, or otherwise opening, the storage vehicle using a puncturing, crushing or equivalent device (operably built into the container, kit, package or pack), or by hand motion, e.g., by twisting or hand turning (e.g., by hand) the container, and thus allowing passage or contact of the contents of the storage vehicle to enter or pass into an ingestible liquid or other edible substance (e.g., an ice cream or a yoghurt), which is also contained within the container, kit, package or pack, which can be initially (before the twisting or turning, puncturing, crushing or otherwise opening) in a separate compartment from the storage compartment. This twisting or turning, or puncturing, crushing or otherwise opening of the storage compartment and the passage or contact of the contents of the storage vehicle to the ingestible liquid effectively places the contents of the storage (e.g., a powder or freeze-dry comprised of or within a pharmaceutical preparation or therapeutic combination as provided herein) into the ingestible liquid or substance, which can be e.g., water, a milk, a yoghurt, an ice cream, a yogurt, a juice (e.g., a fruit juice, an apple juice), an apple sauce, or a masking drink. The container, kit, package or pack can be designed as an infant feeding bottle, e.g., comprising a nipple or teat for the very young.

In alternative embodiments, this simple twisting or turning, or puncturing or crushing device, allows the storage containers, e.g., geltabs or capsules, to be punctured and/or crushed or otherwise “opened”, allowing the contents of the storage container, (e.g., a powder or freeze-dry comprised of or within a pharmaceutical preparation or therapeutic combination as provided herein), to fall out in to the liquid or food compartment, e.g., to the bottom end of a device or straight into a bottle or a container held underneath or configured to be attached and underneath. For example, in this way a provider, e.g., the mother, can purchase a supply of storage containers, e.g., geltabs or capsules, convert them as needed into a powder capable of being mixed a liquid of her choice that the child will be ingesting.

In alternative embodiments, for those capable of swallowing tablets, capsules and the like, the storage containers, e.g., geltabs, tablets or capsules, are manufactured as enteric coated to bypass the acid of the stomach and bile of the duodenum, such that the storage containers, e.g., geltabs, tablets or capsules open (e.g., dissolve) in the jejunum or below.

In alternative embodiments, further provided are instructions for use, e.g., that when emptied into a drink, providers (e.g., the mothers of infants or children) are advised to choose a drink or food that has its own buffering capacity such as flavoured milk, chocolate milk, ice cream, yoghurt, ice blocks, frozen icicles, or simply milk, e.g., that is being fed to the infant or child by a bottle, e.g., a milk bottle, with a nipple or teat.

In alternative embodiments, storage containers, e.g., geltabs, tablets or capsules, or any formulation as provided herein, also comprises an antacid, e.g., a calcium carbonate, magnesium hydroxide, propylene glycol alginate and sodium alginate, or the combination of aluminium hydroxide with magnesium trisilicate, magnesium oxide or magnesium carbonate, so that when the storage container is punctured, crushed or otherwise opened and put into contact with the liquid, e.g., the feeding bottle, and ingested, there will be greater protection from acid damage. In alternative embodiments, methods and instructions further comprise the infant or child also being given an acid suppressant beforehand to permit more viable living bacteria to arrive in the colon.

In alternative embodiments, pharmaceutical preparation or therapeutic combination as provided herein are formulated or manufactured as storage vehicles, e.g., tablets, geltabs, lozenges, pills, capsules and the like; and in alternative embodiments, these storage vehicles are contained in, or contained in a kit with, or packaged with, or sold together with, a storage vehicle ‘cracking’, puncturing, or otherwise opening or releasing device (e.g., as a powder, e.g., as lyophilised material). These can be dispensed together, or configured together, or manufactured together, as a simple way of meeting the needs of both infants, the very young, older children and needful (e.g., handicapped) adults; e.g., as a powder, e.g., as lyophilised material, e.g., from their storage vehicles, e.g., as encapsulated formulations, pharmaceuticals or pharmaceutical preparations, thus permitting successful clinical administration on a frequent, e.g., bid, tid, or daily, basis for prolonged periods.

### Methods of Use and Applications of Devices and Compositions

In alternative embodiments, provided are pharmaceutical preparation or therapeutic combination, devices and methods for treating, ameliorating, reversing, causing the remission of and/or preventing (acting as a prophylaxis) an IBD. In alternative embodiments, provided are pharmaceutical compositions, therapeutic combinations, devices and methods for treating, ameliorating, reversing (e.g., causing or inducing the remission of) and/or preventing (acting as a prophylaxis) an inflammatory bowel disease or an inflammatory bowel disorder (both collectively referred to as IBD), Ulcerative Colitis; Crohn's disease; J-pouch; fistulising Crohn's disease; a Colitis which can be microscopic, lymphocytic or collagenous; an eosinophilic colitis; indeterminate colitis; idiopathic colitis; diverticulosis and diverticulitis; relapsing diverticulitis; constipation associated inflammatory bowel disease and/or small intestinal bacterial overgrowth; Irritable Bowel Syndrome (IBS) with or without diarrhoea, constipation or pain predominant IBS; periodontitis; rheumatoid arthritis; respiratory infections, appendicitis, vascular disorders such as thrombophlebitis; bacteremia; osteomyelitis; septic shock; Alzheimer's disease; Lemierre syndrome (postanginal sepsis); colonic polyps or adenomas (optionally hyperplastic, adenomatous or serrated adenomas) or preventing the growth of, or slowing the progression or recurrence of, colonic polyps or adenomas, bowl cancer, or metastases (optionally preventing the initiation, promotion or recurrence of bowl cancer or metastasis); pharyngitis; otitis; sinusitis; and any disease, symptom or condition caused or exacerbated by a Fusobacteria, e.g., a F. nucleatum or F. varium infection. In alternative embodiments, pharmaceutical preparation or therapeutic combination and methods as provided herein can be used effectively for treating, ameliorating, reversing, causing the remission of and/or preventing (acting as a prophylaxis) conditions associated with any of the above-referenced infections, diseases or conditions.

### Multicomponent Packaging

Provided are multi-component delivery systems, e.g., products of manufacture, comprising e.g., a pharmaceutical preparation or therapeutic combination as provided herein or used to practice methods as provided herein, e.g., formulated and dosaged for oral administration as a powder, e.g., a lyophilised powder, and another component, e.g., a liquid; these multi-component delivery systems, e.g., products of manufacture, can be designed or manufactured as described e.g., in U.S. Pat. Nos. 8,968,717; 8,931,665; 7,861,854; 7,018,089; 6,626,912; and, U.S. Pat. App. Pub nos. 2010/0034574; 2009/0180923; 20090232886; 2008/0160076; 2007/0087048; 2007/0036830; 2007/0074979; 2005/0205438; 2004/0089563.

### Packaging

Provided are compositions, including preparations, pharmaceutical preparation or therapeutic combination, formulations and/or kits, comprising combinations of ingredients, as described herein. In alternative embodiments, these combinations can be mixed and administered together, or alternatively, they can be an individual member of a packaged combination of ingredients, e.g., a liquid component and a solid product component manufactured in a separate compartment, package, kit or container; e.g., where all or a subset of the combinations of ingredients are manufactured in a separate compartment, package or container. In alternative aspects, the package, kit or container comprises a blister package, a clamshell, a tray, a shrink wrap and the like.

In one aspect, the package, kit or container comprises a “blister package” (also called a blister pack, or bubble pack). In one aspect, the blister package is made up of two separate elements: a transparent plastic cavity shaped to the product and its blister board backing. These two elements are then joined together with a heat sealing process which allows the product to be hung or displayed. Exemplary types of “blister packages” include: Face seal blister packages, gang run blister packages, mock blister packages, interactive blister packages, slide blister packages.

Blister packs, clamshells or trays are forms of packaging used for goods; thus, provided are for blister packs, clamshells or trays comprising a formulations, pharmaceutical preparations or pharmaceutical compositions used to practice methods as provided herein. Blister packs, clamshells or trays can be designed to be non-reclosable, so consumers can tell if a package has already opened. They are used to package for sale goods where product tampering is a consideration, such as the pharmaceuticals as provided herein. In one aspect, a blister pack comprises a moulded PVC base, with raised areas (the “blisters”) to contain the tablets, pills, etc. comprising the combinations of formulations, pharmaceutical preparations or pharmaceutical compositions as provided herein, covered by a foil laminate. Tablets, pills, etc. are removed from the pack either by peeling the foil back or by pushing the blister to force the tablet to break the foil. In one aspect, a specialized form of a blister pack is a strip pack. In one aspect, in the United Kingdom, blister packs adhere to British Standard 8404.

In one embodiment, provided is a method of packaging wherein the compositions comprising combinations of ingredients are contained in-between a card and a clear PVC. The PVC can be transparent so the item (pill, tablet, geltab, etc.) can be seen and examined easily; and in one aspect, can be vacuum-formed around a mould so it can contain the item snugly and have room to be opened upon purchase. In one aspect, the card is brightly colored and designed depending on the item (pill, tablet, geltab, etc.) inside, and the PVC is affixed to the card using pre-formed tabs where the adhesive is placed. The adhesive can be strong enough so that the pack may hang on a peg, but weak enough so that this way one can tear open the join and access the item. Sometimes with large items or multiple enclosed pills, tablets, geltabs, etc., the card has a perforated window for access. In one aspect, more secure blister packs, e.g., for items such as pills, tablets, geltabs, etc. are used, and they can comprise of two vacuum-formed PVC sheets meshed together at the edges, with the informative card inside. These can be hard to open by hand, so a pair of scissors or a sharp knife may be required to open.

In one aspect, blister packaging comprises at least two or three or more components: a thermoformed “blister” which houses multi-ingredient combination as provided herein, and then a “blister card” that is a printed card with an adhesive coating on the front surface. During the assembly process, the blister component, which is most commonly made out of PVC, is attached to the blister card using a blister machine. This machine introduces heat to the flange area of the blister which activates the glue on the card in that specific area and ultimately secures the PVG blister to the printed blister card. The thermoformed PVG blister and the printed blister card can be as small or as large as you would like, but there are limitations and cost considerations in going to an oversized blister card. Conventional blister packs can also be sealed (e.g., using an AERGO 8 DUO™, SCA Consumer Packaging, Inc., DeKalb Ill.) using regular heat seal tooling. This alternative aspect, using heat seal tooling, can seal common types of thermoformed packaging.

In alternative embodiments, formulations, pharmaceutical preparations or therapeutic combinations, pharmaceutical preparations or pharmaceutical compositions are formulated, e.g., as a powder, e.g., as lyophilised material, e.g., a lyophilized encapsulated product, e.g., for practicing methods as provided herein, can be packaged alone or in combinations, e.g., as “blister packages” or as a plurality of packettes, including as lidded blister packages, lidded blister or blister card or packets or packettes, or a shrink wrap.

In alternative embodiments, laminated aluminium foil blister packs are used, e.g., for the preparation of a pharmaceutical preparation or therapeutic combination, formulations, pharmaceutical preparations or pharmaceutical compositions as provided herein. Products or kits comprise an aqueous solution(s) which are dispensed (e.g., by measured dose) into containers. Trays can be freeze-dried to form tablets which take the shape of the blister pockets. The alufoil laminate of both the tray and lid fully protects any highly hygroscopic and/or sensitive individual doses. In one aspect, the pack incorporates a child-proof peel open security laminate. In one aspect, the system gives tablets an identification mark by embossing a design into the alufoil pocket that is taken up by the tablets when they change from aqueous to solid state. In one aspect, individual ‘push-through’ blister packs/packettes are used, e.g., using hard temper aluminium (e.g., alufoil) lidding material. In one aspect, hermetically-sealed high barrier aluminium (e.g., alufoil) laminates are used. In one aspect, products of manufacture provided herein include kits or blister packs, use foil laminations and strip packs, stick packs, sachets and pouches, peelable and non-peelable laminations combining foil, paper, or film for high barrier packaging.

In alternative embodiments, multi-component products of manufacture, including kits or blister packs as provided herein, include memory aids to help remind patients when and how to take the therapeutic agent. This safeguards the therapeutic agent's efficacy by protecting each tablet, geltab or pill until it's taken; gives the product or kit portability, makes it easy to take a dose anytime or anywhere.

### EXAMPLES

### Example 1

A 41-year-old female patient with a 12 year history of ulcerative colitis presented with 4-15 diarrhoeal stools every day and 2-3 at night. She had been treated with anti-inflammatory medications (including mesalazine, azathioprine, and prednisone) and these were not achieving more than a transient response. She continued to have bleeding urgency and occasional episodes of incontinence. Her initial colonoscopy showed pancolitis.

She was commenced on secnidazole (400 mg three times daily) combined with rifampicin (increasing from 150 mg twice daily to 300 mg twice daily after four weeks), together with doxycycline (50 mg twice daily).

Over the next 6 to 8 weeks her frequent motions slowly reduced in frequency to 3-6/d, bleeding was no longer visible and urgency had improved quite dramatically. She then continued on the same regimen a further six months when a colonoscopy was repeated. The previous pancolitis now improved markedly with almost complete healing of the inflamed mucosa. Biopsy showed areas absent of colitis, and some chronic colitis regions. The appearance was that of a normal colon.

### Example 2

A 42 year old male patient, with a 4 year history of Crohn's disease, presented with a Crohn's Disease Activity Index (CDAI) score of 550, 7-10 liquid stools daily, abdominal pain, inflammation, and deep ulceration under scope. The patient had previous exposure to anti-TNF therapy, which had been only transiently effective.

The patient was commenced on a combination of rifaximin (500 mg bid), tinidazole (500 mg bid), and nitazoxanide (500 mg bid). Dosage of each drug was increased 2 weeks later by 500 mg, with rifaximin slowly increased to a final dosage of 1.5 g big (total 3 g daily).

After 4 weeks, the patient reported a marked reduction in liquid stools and abdominal pain. He had a review colonoscopy at 5 months showing excellent healing of ulcers and marked improvement of inflammation. After another 4 months treatment the patient reported more normal stool frequency of approximately 3 soft formed stools a day, soft formed and absence of abdominal pain. Colonoscopy 2 months later showed near complete ulcer healing and minor inflammation. The patient's CDAI score was 120.

### Example 3

A 32-year-old male with long-standing ulcerative colitis (UC) extending for 45 cm from the anus presented for review as his original treatments with immunomodulators were failing to control his UC. He was colonoscoped and it was found that the inflammatory process was confluent, starting at the anus reaching to about 40 cm. Cultures and biopsies were collected. He had been treated with azathioprine, and mesalazine plus steroids, but had failed Humira treatment.

He was commenced on a combination of fosfomycin (1 g twice daily) with doxycycline (50 mg twice daily) and metronidazole (400 mg twice daily), for 4 weeks.

He had a clear improvement in his frequency of bloody stools after 2 weeks, and by 4 weeks of treatment his stools became formed. Bleeding ceased, after persisting for about a year on previous medications. At his 8 week colonoscopy there were still minute spots of inflammation but generally the improvement was marked. With ongoing additional fecal microbiota transplant capsule treatment the colon became normal, at the 16 week colonoscopy being indistinguishable from normal bowel.

A number of embodiments of the invention have been described. Nevertheless, it will be understood that various modifications may be made without departing from the spirit and scope of the invention. Accordingly, other embodiments are within the scope of the following claims.

1. A method for:
treating, ameliorating, reversing, causing the remission of, and/or preventing or acting as a prophylaxis, or preventing the initiation of, an inflammatory bowel disease or disorder (IBD); ulcerative colitis; Crohn's disease; J-pouch; fistulising Crohn's disease; a colitis which can be microscopic, lymphocytic or collagenous; an eosinophilic colitis; indeterminate colitis; idiopathic colitis; diverticulosis; diverticulitis; relapsing diverticulitis; constipation associated inflammatory bowel disease and/or small intestinal bacterial overgrowth; irritable bowel syndrome (IBS) with or without diarrhoea, constipation or pain predominant IBS; periodontitis; rheumatoid arthritis; respiratory infections, appendicitis, vascular disorders such as thrombophlebitis; bacteremia; osteomyelitis; septic shock; Alzheimer's disease; Lemierre syndrome or postanginal sepsis; colonic polyps; or adenomas (optionally hyperplastic, adenomatous or serrated adenomas); or
preventing the growth of, or slowing the progression or recurrence of, colonic polyps or adenomas, bowl cancer, or metastases optionally preventing the initiation of or recurrence of or promotion of bowl cancer or metastasis; pharyngitis; otitis; or sinusitis; or
treating, ameliorating, reversing, causing the remission of, and/or preventing any disease, symptom or condition caused or exacerbated by a Fusobacteria infection, e.g., a F. nucleatum, F. varium, F. simae, F. periodonticum, F. equimun, or F . necrogenes) infection,
in an individual in need thereof, comprising administering to the individual in need thereof, optionally, a human or animal, a formulation, a pharmaceutical preparation, a therapeutic combination, or a pharmaceutical composition comprising or consisting of:
(a) (i) a rifaximin (or, In-25-yl acetate; (16Z,18E,28E)-5,6,21,23-tetrahydroxy-27-methoxy-2,4,11,16,20,22,24,26-octamethyl-1,15-dioxo-1,2-dihydro-2,7-(epoxypentadeca[1,11,13]trienoimino)furo[2″,3″:7′,8′]naphtho[1′,2′:4,5]imidazo[1,2-a]pyrid; or, 2S-Acetyloxy-5,6,21,23-tetrahydroxy-27-methoxy-2,4,11, 16,20,22,24,26-octamethyl-2,7-(epoxypentoeleca(1,11,13)trienimino)benzofuro[4,5-e]pyride[1,2-a]benzimidazole-1,15(2H)-dione; 80621-81-4; or, enantiomers or stereoisomers thereof) (optionally a XIFAXAN™, XIFAXANTA™, RITACOL™, FATROXIMIN™, XIFAXSAN™, RIFAXIMINUM™, RIFAXIMINUN™, RIFAXIMINE™, RIFAXIMIN™, RIFAXIDIN™, RIFAXIMINA™, RIFAMYCIN™, VETRANAL™, or NORMIX™), a polymorphic form of a rifaximin or a rifaximin equivalent thereof, an extended intestinal release (EIR) rifaximin, a rifamycin derivative, a rifampicin (or rifampin) (optionally RIFADIN™), a rifabutin (optionally MYCOBUTIN™), a rifapentin (optionally PRIFTIN™), a rifalazil, a bicozamycin, a pyrido-imidazo rifamycin, a dehydro-rifaximin, a rifaximin histidine, a rifaximin tryptophan, an 11-desmethyl-rifaximin (optionally an 11-desmethyl NORMIX™), a rifaximin beta-cyclodextrin, fosfomycin, or equivalents thereof or a mixture or a combination thereof,
(ii) vancomycin, neomycin, tobramycin, paromomycin or streptomycin,
(iii) gentamicin, streptomycin, ridinilazole, teicoplanin, streptomycin and neomycin, fidaxomicin, ramoplanin, surotomycin, capozide, a partially absorbed agent, optionally comprising a tinidazole, metronidazole, nitazoxanide, amoxicillin, tetracyclines, ornidazole, secnidazole, ciprofloxacin or an ansamycin, or
(iv) an antibiotic or drug as listed in Table 1; or
(b) an antibiotic or drug of (a), and at least one additional antimicrobial or antibiotic agent,
wherein optionally for (a) or (b) the rifaximin or rifaximin polymorphic form thereof or rifaximin equivalent comprises:
(i) a rifaximin, a rifaximin polymorph or a rifaximin equivalent as described in U.S. Pat. No. 9,273,066, optionally comprising a polymorphic form zeta of rifaximin exhibiting an X-ray powder diffraction pattern having characteristic peaks expressed in degrees 2 theta (+/−0.20 degree theta) comprising 4.69, 7.63, 12.52, 13.87;
(ii) a 25-desacetyl rifaximin, or a rifaximin, a rifaximin polymorph or a rifaximin equivalent, as described in U.S. Pat. No. 9,364,467, optionally comprising a 25-desacetyl rifaximin or a pharmaceutically acceptable salt thereof (optionally a sodium, potassium, calcium, magnesium, ammonium, or chlorine pharmaceutically acceptable salt of 25-desacetyl rifaximin), wherein optionally the 25-desacetyl rifaximin has the formula: 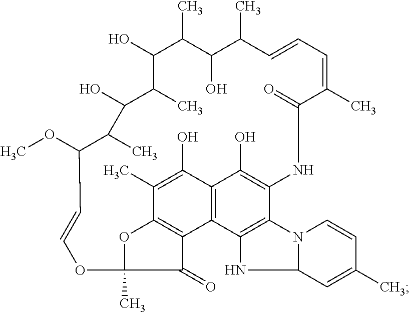 [SMILES: COC1/C=C/O[C@@]2(C)Oc3c(C)c(O)c4c(O)c(c5c(c4c3C2=O)NC2C=C(C)C=CN52)NC(=O)/C(C)=C\C=C\C(C)C(O)C(C)C(O)C(C)C(O)C1C]
(iii) a rifaximin, a rifaximin polymorph or a rifaximin equivalent as described in U.S. Pat. No. 9,546,183, optionally comprising a polymorphic Form B of rifaximin exhibiting an X-ray powder diffraction pattern having characteristic peaks expressed in degrees 2 theta (+/−0.20 degree theta) at 5.24, 6.84, 7.74, 8.71, 10.16, and 12.21,
(iv) a rifaximin amorphous form or a rifaximin equivalent as described in U.S. Pat. No. 9,700,545, optionally comprising an amorphous form of rifaximin exhibiting an X-ray powder diffraction pattern having characteristic peaks expressed in degrees:
(v) 2 theta (+/−0.20 degree theta) at 7.3, 11.3-17.8, and 15.8 degrees 2 theta; 2 theta (+/−0.20 degree theta) at 5.1-10.1, 11.3-17.8, and 15.8 degrees 2 theta; or (3) 2 theta (+/−0.20 degree theta) at 5.1-10.1, 7.3, and 11.3-17.8 degrees 2 theta,
(v) a rifaximin, a rifaximin polymorph or a rifaximin equivalent as described in U.S. Pat. No. 9,359,374, or U.S. Pat. No. 9,725,466, optionally comprising a polymorphic form APO-III of rifaximin characterized by a PXRD diffractogram comprising peaks, in terms of degrees 2-theta, at approximately 7.1, 8.4, 11.6, 13.1, 18.5, 18.8, and 25.0,
(vi) a rifaximin, a rifaximin polymorph or a rifaximin equivalent as described in U.S. Pat. No. 9,421,195,
(vii) a rifaximin, a rifaximin polymorph or a rifaximin equivalent as described in U.S. Pat. No. 7,045,620, optionally a crystalline polymorphous form of a rifaximin, a rifaximin polymorph or a rifaximin equivalent; and/or
(viii) a controlled-release or spray-dried rifaximin, rifaximin polymorph or rifaximin equivalent as described in U.S. Pat. No. 9,498,442, optionally rifaximin, rifaximin polymorph or rifaximin equivalent characterized by an X-Ray diffraction spectrum showing diffraction halo peaks in the range 7.75 degree+−0.0.2-18.33 degree±0.0.2, 2 theta, with maximum at about 7.75 degree±0.0.2 and in the range 14.54 degree±0.0.2 and 18.33 degree±0.0.2, 2 theta.

2. The method of claim 1, wherein the IBD further comprises or is associated with a condition or side effect comprising diarrhoea, rectal bleeding, mucus, urgency, incontinence, nocturnal diarrhoea; together with lower abdominal pain, weight loss, excessive gas production, bloating, loss of appetite, joint pains/symptoms, and optionally administration of the formulation, pharmaceutical preparation, therapeutic combination or pharmaceutical composition treats, ameliorates, reverses, causes the remission of, and/or prevents or acts as a prophylaxis one, several or all of these conditions or side effects.

3. The method of claim 1, wherein the formulation, pharmaceutical preparation, therapeutic combination or pharmaceutical composition further comprises at least one additional antimicrobial or antibiotic agent, or further comprises a drug or a probiotic,
and optionally the at least one additional antimicrobial or antibiotic agent comprises a vancomycin, a metronidazole, a tinidazole, an ornidazole, a secnidazole, an antibiotic or drug as listed in Table 1, or a combination thereof,
and optionally the at least one additional antimicrobial or antibiotic agent comprises: an antibiotic or antibacterial agent from one or more of the following classes selected from: tetracyclines, penicillins, macrolides, quinolones, chloramphenicol, rifamycins, sulphonamides, co-trimoxazole, and oxazolidinones,
and optionally the at least one additional antimicrobial or antibiotic agent comprises: a doxycycline, chlortetracycline, tetracycline hydrochloride, oxytetracycline, demeclocycline, methacycline, minocycline, penicillin, amoxycillin, erythromycin, clarithromycin, roxithromycin, azithromycin, spiramycin, oleandomycin, iosamycin, kitsamysin, flurithromycin, nalidixic acid, oxolinic acid, norfloxacin, perfloxacin, am ifloxacin, ofloxacin, moxifloxacin, ciprofloxacin, sparfloxacin, levofloxacin, rifabutin, rifampicin, rifapentin, rifalazil, sulfisoxazole, sulfamethoxazole, sulfadiazine, sulfadoxine, sulfasalazine, sulfaphenazole, dapsone, sulfacytidine, linezolid or any combination thereof
and optionally the at least one additional antimicrobial or antibiotic agent comprises:
an ampicillin, a sulbactama tetracycline, a cephalosporin, a carbapenem, an imipenem, a meropenem, a monobactam, a lincosamide, a clindamycin, a quinolone, a fluoroquinolone, a sulphonamide, a fradicin, a nitroimidazole, a metronidazole, a tinidazole, a secnidazole, an anti-Clostridial agent, or a ramoplanan,
an aminoglycoside antibiotic, a gentamycin, a neomycin, a streptomycin, a paromomycin, a verdamicin, a mutamicin, a sisomicin, a netilmicin, a retymicin, a kanamycin, an amphenicol, an ansamycin, a beta-lactam (β-lactam) antibiotic, a carbapenem, a cephalosporin, a cephamycin, a monobactam, an oxacephem, a lincosamide antibiotic, a clindamycin, or a lincomycin,
a glycopeptide antibiotic, a vancomycin, a teicoplanin, a telavancin, a bleomycin, a ramoplanin, a decaplanin, a polypeptide antibiotic, an actinomycin, an actinomycin D, a bacitracin, a bacitracin, a tetracycline, a 2,4-diaminopyrimidine class antibiotic, a clavacin, a clairformin, a claviform, an expansine, a clavatin, an expansin, a gigantin, a leucopin, a patuline or a patulin), or
an equivalent thereof or a combination thereof,
and optionally the at least one additional antimicrobial or antibiotic agent comprises:
(i) a rifaximin, a polymorphic form of a rifaximin or a rifaximin equivalent thereof, an extended intestinal release (EIR) rifaximin, a rifamycin derivative, a rifampicin (or rifampin), a rifabutin, a rifapentin, a rifalazil, a bicozamycin, a pyrido-imidazo rifamycin, a dehydro-rifaximin, a rifaximin histidine, a rifaximin tryptophan, an 11-desmethyl-rifaximin, a rifaximin beta-cyclodextrin, or equivalents thereof or a mixture or a combination thereof,
(ii) vancomycin, neomycin, tobramycin, paromomycin or streptomycin,
(iii) gentamicin, streptomycin, ridinilazole, teicoplanin, streptomycin and neomycin, fidaxomicin, ramoplanin, surotomycin, capozide, a partially absorbed agent, optionally comprising a tinidazole, metronidazole, nitazoxanide, amoxicillin, tetracyclines, ornidazole, secnidazole, ciprofloxacin or an ansamycin,
(iv) an antibiotic or drug as listed in Table 1, or
(v) any combination thereof,
and optionally the antimicrobial or antibiotic agent comprises a three-drug therapeutic combination comprising: rifaximin, tinidazole and nitazoxanide; rifaximin, tinidazole and oral tobramycin; rifaximin, amoxicillin and metronidazole; rifaximin, paromomycin and nitazoxanide; rifaximin, paromomycin and vancomycin; rifaximin, tinidazole and paromomycin; rifaximin, ridinilazole and tobramycin; rifaximin, ridinilazole and paromomycin; or rifaximin, ridinilazole and nitazoxanide;
and optionally teicoplanin is substituted for any one of the second or third drug in the 3-drug therapeutic combination.

4-9. (canceled)

10. The method of 1, wherein the formulation, pharmaceutical preparation, therapeutic combination, or pharmaceutical composition comprises:
(a) a rifamycin, a nitroimidazole, and a tetracycline antibiotic,
and optionally the rifamycin is rifampicin, the nitroimidazole is secnidazole, and the tetracycline antibiotic is doxycycline;
(b) a rifamycin, a nitroimidazole, and a thiazolide,
and optionally the rifamycin is rifaximin, the nitroimidazole is tinidazole, and the thiazolide is nitazoxanide; or
(c) fosfomycin, a nitroimidazole, and a tetracycline antibiotic,
and optionally the nitroimidazole is metronidazole, and the tetracycline antibiotic is doxycycline.

11-15. (canceled)

16. The method of claim 1, wherein:
(a) the individual exhibits at least an about 5% to 10%, or 10% to 20%, or 20% to 30%, or 30% to 40%, or 40% to 50%, or 50% to 60%, or 70% to 80%, or 80% to 90%, or substantially complete, reduction in IBD symptoms or side effects or severity after administration of the formulation, pharmaceutical preparation, therapeutic combination or pharmaceutical composition to the individual in need thereof as compared to before initiating the administration,
(b) the at least an about 5% to 10%, or 10% to 20%, or 20% to 30%, or 30% to 40%, or 40% to 50%, or 50% to 60%, or 70% to 80%, or 80% to 90%, or substantially complete, reduction in IBD symptoms or side effects or severity is achieved after about 1, 2 or 3 or more weeks, or after about 1 to 2 months, or after about 2 to 6 months, of initiating the administration; or
(c) the at least an about 5% to 10%, or 10% to 20%, or 20% to 30%, or 30% to 40%, or 40% to 50%, or 50% to 60%, or 70% to 80%, or 80% to 90%, or substantially complete, reduction in IBD symptoms or side effects or severity is maintained for at least about 4 to 8 weeks, or 2 to 6 months, after discontinuing the administration to the individual.

17-18. (canceled)

19. The method of claim 1, wherein the formulation, pharmaceutical preparation, therapeutic combination or pharmaceutical composition is formulated as a chewable delivery vehicle, a gum, a gummy, a candy, a lozenge, an ice cream or an ice, a yogurt or a drink.

20. The method of claim 1, wherein:
(a) a unit dosage of the formulation, pharmaceutical preparation, therapeutic combination or pharmaceutical composition is a pediatric unit dosage, and optionally the unit dosage is between about 10 mg and 1100 mgm, or between about between about 40 mg and 4,000 mgm, or is about 10, 20, 30, 40, 50, 60, 70, 75, 80, 90, 100, 125, 150, 175, 200, 225, 250, 275, 300, 325, 350, 375, 400, 425, 450, 475, 500, 600, 700, 750, 800, 900, 1000, 1100, 1500, 2000, 2500, 3000, 3500, 4000, or more mg per unit dose, which optionally can be administered once a day, bid or tid, or a four times a day, five times a day or six times a day or more, regimen;
(b) a daily dosage of the formulation, pharmaceutical preparation, therapeutic combination or pharmaceutical composition is about 50, 75, 100, 125, 150, 175, 200, 225, 250, 275, 300, 325, 350, 375, 400, 425, 450, 475, 500, 600, 700, 750, 800, 900, 1000, 1100, 1500, 2000, 2500, 3000, 3500, 4000, or more mg per day, or between about 100 and 1100 mgm per day total, or between about 400 and 4000 mg per day, which optionally can be administered in a once a day, bid or tid, or four times a day, five times a day or six times a day or more, regimen;
(c) a unit dosage of the formulation, pharmaceutical preparation, therapeutic combination or pharmaceutical composition is set for or the daily dosage is set for: bid twice a day, tid three times a day, four times a day, five times a day or six times a day or more, with the unit dosage and daily dosage adjusted to be: about 1000 mg/70 kg a day, or about 14 mg/kg a day, for an adult median dose per day; or for a pediatric dosage about 350 mg/25 kg a day, or about 15 to 16 mg/kg, a day; or equivalent;
(d) the daily dosage of the formulation, pharmaceutical preparation, therapeutic combination or pharmaceutical composition is about 25 mg to 20 grams (gm) bid, or about 400, 425, 450, 475, 500, 600, 700, 750, 800, 900, 1000, 1100, 1200, 1500, 2000, 2500, 3000, 3500, 4000, or more mgm once a day, bid or tid, or four times a day, five times a day or six times a day or more;
(e) the daily dosage of the formulation, pharmaceutical preparation, therapeutic combination or pharmaceutical composition, or one ingredient of the formulation, pharmaceutical preparation, therapeutic combination or pharmaceutical composition, is increased or “ramped up” every week, or every other week, by about 25, 50, 75, 100, 125, 150, 175, 200, 225, 250, 275, 300, 325, 350, 375, 400, 425, 450, 475, 1500, 2000, 2500, 3000, 3500, 4000, or more or more mg per week, or every other week,
and optionally this “ramping up” or increasing of dosages continues for about a month, about 6 months or about a year, or until symptoms of IBD significantly diminish or abate, or significantly diminish or abate without need for administration of the formulation, the pharmaceutical or the pharmaceutical preparation.

21-24. (canceled)

25. The method of claim 1, wherein the formulation, the pharmaceutical or the pharmaceutical preparation further comprises a flavoring or a sweetening agent, an aspartamine, a stevia, monk fruit, a sucralose, a saccharin, a cyclamate, a xylitol, a vanilla, an artificial vanilla or chocolate or strawberry flavor, an artificial chocolate essence, or a mixture or combination thereof.

26. The method of claim 1, wherein the formulation, pharmaceutical preparation, therapeutic combination or pharmaceutical composition further comprises a preservative, a benzoic acid or a potassium sorbate.

27. The method of claim 1, wherein the formulation, pharmaceutical preparation, therapeutic combination or pharmaceutical composition further comprises, or has added to: at least one probiotic or prebiotic,
wherein optionally the prebiotic comprises an inulin, lactulose, extracts of artichoke, chicory root, oats, barley, various legumes, garlic, kale, beans or flacks or an herb,
and optionally the probiotic comprises a cultured or stool-extracted microorganism or bacteria, or a bacterial component, and optionally the probiotic bacteria or bacterial component comprises or is derived from a non-pathogenic Clostridia, a Bacteroidetes, a Firmicutes, a Lactobacilli, a Bifidobacteria, an E. coli, a Strep fecalis, an Actinobacteria, a Proteobacteria, a Verruco-microbia, a Fusobacteria, a Cyanobacteria, a Spirochetes and a Lentisphaerae, and equivalents.

28. The method of claim 1, wherein the formulation, pharmaceutical preparation, therapeutic combination or pharmaceutical composition further comprises, or has added to: at least one congealing agent, wherein optionally the congealing agent comprises an arrowroot or a plant starch, a powdered flour, a powdered potato or potato starch, an absorbant polymer, an Absorbable Modified Polymer, and/or a corn flour or a corn starch.

29. The method of claim 1, wherein the formulation, pharmaceutical preparation, therapeutic combination or pharmaceutical composition further comprises an additive selected from one or more of a saline, a media, a defoaming agent, a surfactant agent, a lubricant, an acid neutralizer, a marker, a cell marker, a drug, an antibiotic, a contrast agent, a dispersal agent, a buffer or a buffering agent, a sweetening agent, a debittering agent, a flavoring agent, a pH stabilizer, an acidifying agent, a preservative, a desweetening agent and/or coloring agent, vitamin, mineral and/or dietary supplement, or a prebiotic nutrient.

30. The method of claim 1, wherein the formulation, pharmaceutical preparation, therapeutic combination or pharmaceutical composition further comprises, or has added to: at least one Biofilm Disrupting Compound, wherein optionally the biofilm disrupting compound comprises an enzyme, a deoxyribonuclease (DNase), N-acetylcysteine, an auranofin, an alginate lyase, glycoside hydrolase dispersin B; a Quorum-sensing inhibitor, a ribonucleic acid III inhibiting peptide, Salvadora persica extracts, Competence-stimulating peptide, Patulin and penicillic acid; peptides—cathelicidin-derived peptides, small lytic peptide, PTP-7, Nitric oxide, neo-emulsions; ozone, lytic bacteriophages, lactoferrin, xylitol hydrogel, synthetic iron chelators, cranberry components, curcumin, silver nanoparticles, Acetyl-11-keto-β-boswellic acid (AKBA), barley coffee components, probiotics, sinefungin, S-adenosylmethionine, S-adenosyl-homocysteine, Delisea furanones, N-sulfonyl homoserine lactones or any combination thereof.

31. The method of claim 1, wherein the formulation, pharmaceutical preparation, therapeutic combination or pharmaceutical composition is formulated as a delayed or gradual enteric release composition or formulation, and optionally the formulation comprises a gastro-resistant coating designed to dissolve at a pH of 7 in the terminal ileum, e.g., an active ingredient is coated with an acrylic based resin or equivalent, e.g., a poly(meth)acrylate, e.g. a methacrylic acid copolymer B, NF, which dissolves at pH 7 or greater, e.g., comprises a multimatrix (MMX) formulation.

32. The method of claim 1, wherein the formulation, pharmaceutical preparation, therapeutic combination or pharmaceutical composition is contained in a delivery vehicle, product of manufacture, container, syringe, device or bag.

33. The method of claim 1, wherein the formulation, pharmaceutical preparation, therapeutic combination or pharmaceutical composition is initially manufactured or formulated as a liquid, a suspension, a gel, a geltab, a semisolid, a tablet, a sachet, a lozenge or a capsule, or as an enteral formulation, or re-formulated for final delivery as a liquid, a suspension, a gel, a geltab, a semisolid, a tablet, a sachet, a lozenge or a capsule, or as an enteral formulation.

34. A product of manufacture comprising or having contained therein a formulation, pharmaceutical preparation, therapeutic combination or pharmaceutical composition of claim 1,
wherein optionally the product of manufacture is an implant or a kit.

35-36. (canceled)
